(19) United States
(12) Patent Application Publication
(10) Pub. No.: US 2006/0024696 A1
(43) Pub. Date: Feb. 2, 2006

(54) Nucleic acid and polypeptide sequences from Lawsonia intracellularis and methods of using

(76) Inventors: Vivek Kapur, Shoreview, MN (US); Connie J. Gebhart, Maple Grove, MN (US)

Correspondence Address: FISH & RICHARDSON P.C., PO BOX 1022, MINNEAPOLIS, MN, 55440-1022, US

(21) Appl. No.: 11/098,686

(22) Filed: Apr. 4, 2005

Related U.S. Application Data
(63) Continuation of application No. PCT/US03/31318, filed on Oct. 1, 2003.
(60) Provisional application No. 60/416,395, filed on Oct. 4, 2002.

(51) Int. Cl. C12Q 1/68; C07H 21/04; C12P 21/06; C07K 14/195; C12N 15/74

(52) U.S. Cl. 435/6; 435/69.1; 435/252.3; 435/320.1; 530/350; 536/23.7

(57) ABSTRACT

The present invention provides nucleic acid molecules unique to L. intracellularis. The invention also provides the polypeptides encoded by the L. intracellularis-specific nucleic acid molecules of the invention, and antibodies having specific binding affinity for the polypeptides encoded by the L. intracellularis-specific nucleic acid molecules. The invention further provides for methods of detecting L. intracellularis in a sample using nucleic acid molecules, polypeptides, and antibodies of the invention. The invention additionally provides methods of preventing a L. intracellularis infection in an animal.

### CROSS REFERENCE TO RELATED APPLICATIONS

This application is a continuation and claims benefit of priority to International Application No. PCT/US03/31318, having an International Filing Date of Oct. 1, 2003, which claims benefit under 35 U.S.C. § 119(e) of U.S. Application No. 60/416,395, filed Oct. 4, 2002.

### FEDERALLY SPONSORED RESEARCH OR DEVELOPMENT

The U.S. Government may have certain rights in this invention pursuant to Grant No. 00-52100-9687 from the USDA-CREES-IFAFS research initiative.

### INCORPORATION-BY-REFERENCE & TEXT

The material on the accompanying compact disc is hereby incorporated by reference into this application. The accompanying compact disc contains twenty files; Table 2.txt, Table 3.txt, Table 4.txt, Table 5.txt, Table 10.txt, Table 11.txt, Table 12.txt, Table 13.txt, Table 14.txt, Table 15.txt, Table 16.txt, Table 17.txt, Table 18.txt, Table 19.txt, Table 20.txt, Table 21.txt, Table 22.txt, Table 23.txt, Table 24.txt, and Table 25.txt, which were created on Apr. 4, 2005. The file named Table 2.txt is 58 KB, Table 3.txt is 76 KB, Table 4.txt is 294 KB, Table 5.txt is 2,386 KB, Table 10.txt is 27 KB, Table 11.txt is 39 KB, Table 12.txt is 191 KB, Table 13.txt is 1,424 KB, Table 14.txt is 11 KB, Table 15.txt is 8 KB, Table 16.txt is 33 KB, Table 17.txt is 466 KB, Table 18.txt is 24 KB, Table 19.txt is 29 KB, Table 20.txt is 161 KB, Table 21.txt is 1,284 KB, Table 22.txt is 9 KB, Table 23.txt is 11 KB, Table 24.txt is 57 KB, and Table 25.txt is 459 KB. The files can be accessed using Microsoft Word on a computer that uses Windows OS.

### TECHNICAL FIELD

This invention relates to bacterial nucleic acid and polypeptide sequences, and more particularly to nucleic acid and polypeptide sequences from Lawsonia intracellularis.

### BACKGROUND

Proliferative enteropathy (PE) is an economically important disease of pigs and other animals and has been reported in swine production facilities from throughout the world. In intensively reared pigs, PE can cause major problems due to a failure to gain weight and thrive, but PE also is a cause of sudden death of infected animals. The disease is characterized by the proliferation of intestinal enterocytes, especially in the ileum, that ultimately manifests itself as a gross thickening of the intestinal wall as seen at necropsy.

Reports of proliferative conditions of the intestines of pigs first appeared in 1931. However, it took more than 40 years before the presence of bacteria was described in the proliferative lesions. The identity of these intracellular organisms, however, remained elusive until the development of specific antisera and DNA probes against this agent strongly supported the hypothesis that this organism represented a novel bacterial species. Subsequently, analyses of 16S ribosomal DNA (rDNA) led to the recognition and naming of this intracellular bacterium as a novel organism, L. intracellularis, and classification in the delta subdivision of the Proteobacteria group. Lawsonia shares 91% 16S rDNA sequence homology with Desulfovibrio desulfuricans, a strictly anaerobic sulfate-reducer, and 92% homology with Bilophila wadsworthia. Further insight into the classification of L. intracellularis was provided by the cloning and sequencing of its groE operon. Phylogenetic analysis using the predicted amino acid sequence of the groEL homologs from databases showed that L. intracellularis is taxonomically isolated from other bacteria whose sequences are known. Using these methods, it's nearest relative was shown to be Helicobacter pylori. However, since there were no groEL sequences from Desulfovibrio species present in the databases at that time, a direct comparison between Desulfovibrio species and L. intracellularis could not be made.

L. intracellularis is a unique obligate intracellular bacterium that is cultivable in vitro only in cell culture and requires a specific microaerophilic environment. It is a Gram-negative organism with a single polar flagellum. The morphology of Lawsonia is a typical vibroid-shaped rod 0.3 to 0.4 by 1.5 by 2.0 um. The life cycle of Lawsonia species within infected cells closely resembles that of another obligately intracellular bacterium, Rickettsia tsutsugamushi. Lawsonia species have only been observed to grow and multiply within the cytosol, often in close proximity to cell mitochondria.

In animals, L. intracellularis causes proliferation of intestinal cells, resulting in enteric disease or even death. The disease is responsible for serious economic loss to swine production worldwide. Proliferative intestinal lesions, caused by this organism, have also been described in numerous other species, including hamsters, foals, dogs, deer, fox, rabbits, rats, emus, ostriches and non-human primates. The wide host range of L. intracellularis and the fact that it has been described in primates suggests that it may also be a human pathogen under certain conditions.

Despite the great morbidity, mortality, and economic impact that results from disease due to L. intracellularis, very little is known about the genetic basis for the virulence of this organism. Further, the molecular mechanisms for infection and virulence and the epidemiology of this organism in pigs and other species remain undetermined. Additionally, little is known about the natural physiology of this organism including factors that enable it to colonize the host. Furthermore, accurate and sensitive methods for the routine detection of infected animals are also lacking. For these reasons, it is important to identify L. intracellularis-specific nucleic acids and/or polypeptides.

### SUMMARY

The present invention provides nucleic acid molecules unique to L. intracellularis. The invention also provides polypeptides encoded by the L. intracellularis-specific nucleic acid molecules of the invention, and antibodies having specific binding affinity for the polypeptides encoded by the L. intracellularis-specific nucleic acid molecules. The invention further provides methods of detecting L. intracellularis in a sample using nucleic acid molecules, polypeptides, or antibodies of the invention. The invention additionally provides methods of preventing a L. intracellularis infection in an animal.

In one aspect, the invention provides an isolated nucleic acid, wherein the nucleic acid comprises a nucleic acid molecule of at least 10 nucleotides in length, the molecule having at least 75% sequence identity to SEQ ID NO:8741, or the complement of the molecule, wherein any the molecule that is 10 to 29 nucleotides in length, in combination with an appropriate second nucleic acid molecule, under standard amplification conditions, generates an amplification product from L. intracellularis nucleic acid but does not generate an amplification product from nucleic acid of any of the organisms selected from the group consisting of Homo sapiens, Pseudomonas aeruginosa, Streptomyces viridochromogenes, Mus musculus, Felis catus, and Xanthomonas campestris. The invention provides for an article of manufacture containing such a nucleic acid of the invention.

A nucleic acid of the invention can have at least 75%, at least 80%, at least 85%, at least 90%, at least 95%, or at least 99% sequence identity to any of SEQ ID NO:1-62, 131-8727, 8736-8739, 8741, or 8743.

In another aspect of the invention, there is provided an isolated nucleic acid, wherein the nucleic acid comprises a nucleic acid molecule of at least 10 nucleotides in length, the molecule having at least 75% sequence identity to any of SEQ ID NOs:1-62, 131-8727, 8736-8739, 8741, or 8743, or the complement of any such molecule, wherein any the molecule that is 10 to N nucleotides in length, in combination with an appropriate second nucleic acid molecule, under standard amplification conditions, generates an amplification product from L. intracellularis nucleic acid but does not generate an amplification product from nucleic acid of any of the organisms shown in Tables 2, 3, 4, and 5 for each respective SEQ ID NO. The value of N for each SEQ ID NO can also be determined from Tables 2, 3, 4, and 5.

In another aspect, the invention provides for vectors comprising a nucleic acid of the invention. Host cells comprising such a vector are further provided by the invention.

In yet another aspect, the invention provides for isolated polypeptides encoded by the nucleic acids of the invention. For example, the nucleic acid molecules having the sequence of SEQ ID NOs:1-62 can encode a polypeptide having an amino acid sequence of SEQ ID NOs:63-124, respectively, or a nucleic acid molecule having the sequence of SEQ ID NO:8741 can encode a polypeptide having an amino acid sequence of SEQ ID NO:8740. The nucleic acid sequence and the encoded amino acid sequence for predicted open reading frames are shown in Tables 18-21 and 22-25, respectively.

In another aspect, the invention provides articles of manufacture that include one or more polypeptides of the invention. In still another aspect of the invention, there are provided antibodies that have specific binding affinity for a polypeptide of the invention.

In another aspect, the invention provides for methods for detecting the presence or absence of L. intracellularis in a biological sample. Such methods include contacting the biological sample with one or more of the nucleic acids of the invention (e.g., SEQ ID NOs:1-62 and 131-8727) under standard amplification conditions, wherein an amplification product is produced if L. intracellularis nucleic acid is present in the biological sample; and detecting the presence or absence of the amplification product. Generally, the presence of the amplification product indicates the presence of L. intracellularis in the biological sample, and the absence of the amplification product indicates the absence of L. intracellularis in the biological sample. Representative animals from which the biological sample can be derived include pigs, hamsters, foals, dogs, deer, fox, rabbits, rats, emus, ostriches, non-human primates, and humans. Representative biological samples include a fecal sample and a blood sample. Further, representative nucleic acids that can be used in the above-described methods include those having the sequence of SEQ ID NO:8728-8735.

In another aspect, the invention provides methods for detecting the presence or absence of L. intracellularis in a biological sample. Such methods include contacting the biological sample with one or more of the nucleic acids of the invention (e.g., SEQ ID NOs:1-62 and 131-8727) under hybridization conditions, wherein a hybridization complex is produced if L. intracellularis nucleic acid molecules are present in the biological sample; and detecting the presence or absence of the hybridization complex. Generally, the presence of the hybridization complex indicates the presence of L. intracellularis in the biological sample, and the absence of the hybridization complex indicates the absence of L. intracellularis in the biological sample. Typically, nucleic acids present in the biological sample are electrophoretically separated. Such electrophoretically separated nucleic acids can be attached to a solid support. Representative solid supports include nylon membranes and nitrocellulose membranes. Further, one or more nucleic acids can be labeled. Representative biological samples include a fecal sample and a blood sample.

In another aspect, the invention provides methods for detecting the presence or absence of L. intracellularis in a biological sample. Such methods include contacting the biological sample with a polypeptide of the invention (e.g., SEQ ID NOs:63-124 and those shown in Tables 22-25), wherein a polypeptide-antibody complex is produced if an antibody having specific binding affinity for the polypeptide is present in the sample; and detecting the presence or absence of the polypeptide-antibody complex. Typically, the presence of the polypeptide-antibody complex indicates the presence of L. intracellularis in the biological sample, and the absence of the polypeptide-antibody complex indicates the absence of L. intracellularis in the biological sample. Polypeptides used in the above-described method can be attached to a solid support. Further, representative biological samples include a blood sample and a milk sample.

In yet another aspect, the invention provides for methods for detecting the presence or absence of L. intracellularis in a biological sample. Such methods include contacting the biological sample with an antibody of the invention (e.g., an antibody having specific binding affinity for a polypeptide having an amino acid sequence of SEQ ID NOs:63-124 and those shown in Tables 22-25), wherein an antibody-polypeptide complex is produced if a polypeptide is present in the biological sample for which the antibody has specific binding affinity, and detecting the presence or absence of the antibody-polypeptide complex. Generally, the presence of the antibody-polypeptide complex indicates the presence of L. intracellularis in the biological sample, and the absence of the antibody-polypeptide complex indicates the absence of L. intracellularis in the biological sample. Antibodies used in the above-described methods can be bound to a solid support. Representative biological samples that can be used in the above-described methods include a blood sample and a fecal sample.

In still another aspect of the invention, there are provided methods of preventing infection by L. intracellularis in an animal. Such methods include administering a compound to the animal, wherein the compound comprises a polypeptide of the invention (e.g., SEQ ID NOs:63-124 and those shown in Tables 22-25). Alternatively, such methods include administering a compound to the animal, wherein the compound comprises a nucleic acid of the invention (e.g., a nucleic acid comprising a nucleic acid molecule having at least 75% sequence identity to SEQ ID NOs:1-62 and 131-8727). Typically, the compound immunizes the animal against L. intracellularis.

In another aspect, the invention provides a composition comprising a first oligonucleotide primer and a second oligonucleotide primer, wherein the first oligonucleotide primer and the second oligonucleotide primer are each 10 to 50 nucleotides in length, and wherein the first and second oligonucleotide primers, in the presence of L. intracellularis nucleic acid, generate an amplification product under standard amplification conditions, but do not generate an amplification product in the presence of nucleic acid from an organism other than L. intracellularis. The invention provides articles of manufacture containing such a composition.

In yet another aspect of the invention, there is provided an isolated nucleic acid that comprises a nucleic acid molecule greater than 10 nucleotides in length having at least 75% sequence identity to SEQ ID NO:8741 or to the complement of SEQ ID NO:8741, wherein said molecule hybridizes under stringent conditions with L. intracellularis nucleic acid but does not hybridize with nucleic acid from an organism other than L. intracellularis under the same hybridization conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows the sequences of L. intracellularis-specific nucleic acid molecules (SEQ ID NOs:1-62).

FIG. 2 shows the polypeptide sequences (SEQ ID NOs:63-124) encoded by L. intracellularis-specific nucleic acids. An * indicates a stop codon.

FIG. 3 shows representative nucleic acid molecules having 75%, 80%, 85%, 90%, 95%, and 99% sequence identity to SEQ ID NO:2 (SEQ ID NOs:125-130, respectively).

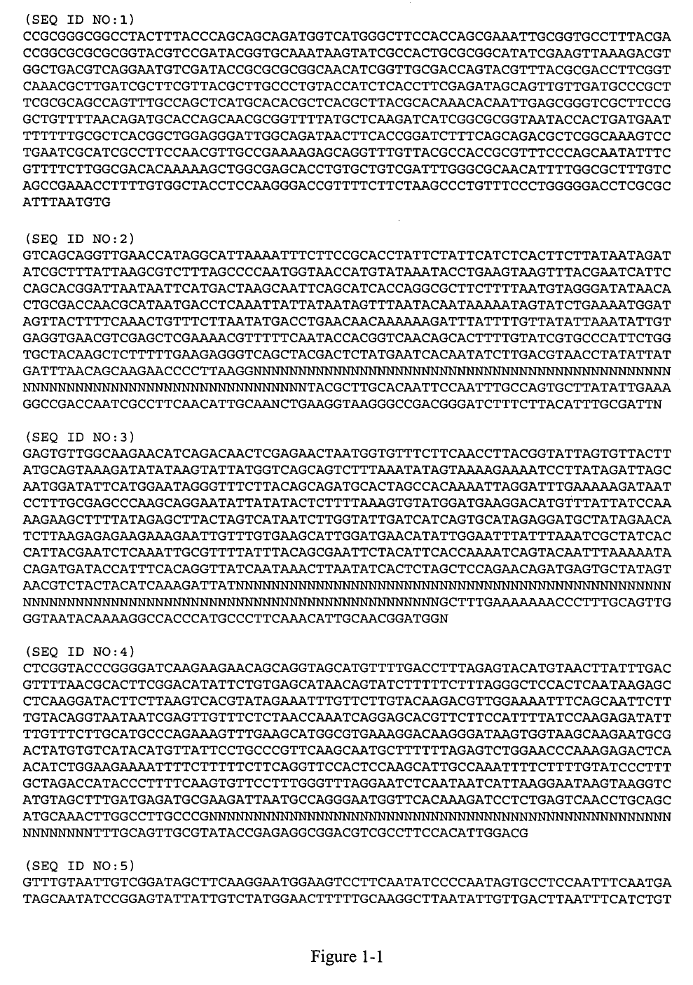

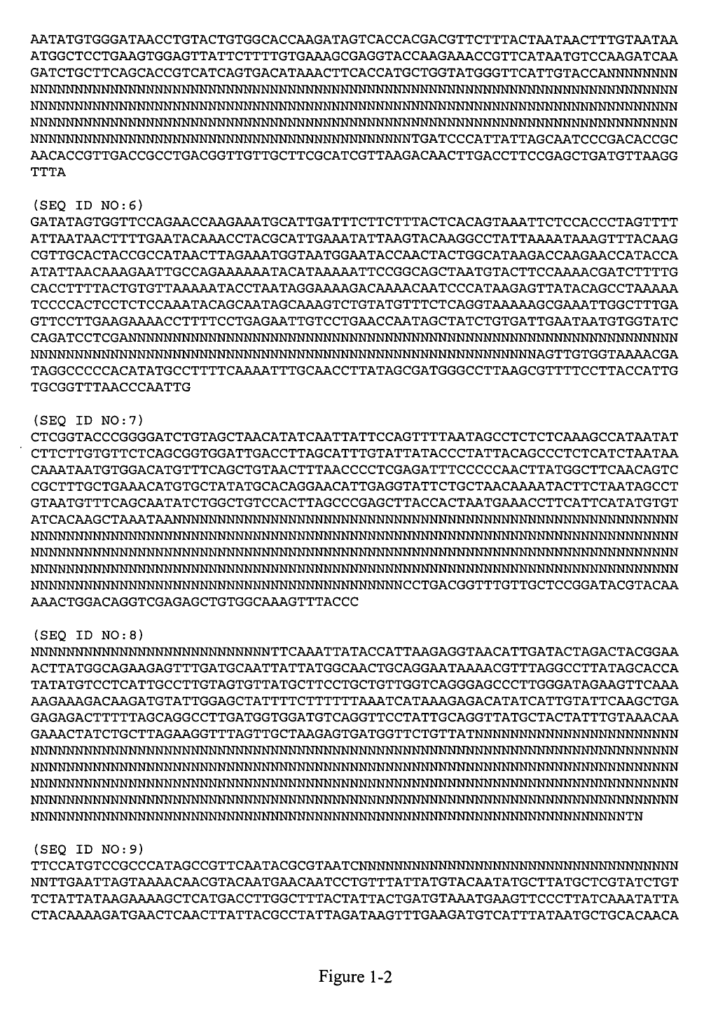

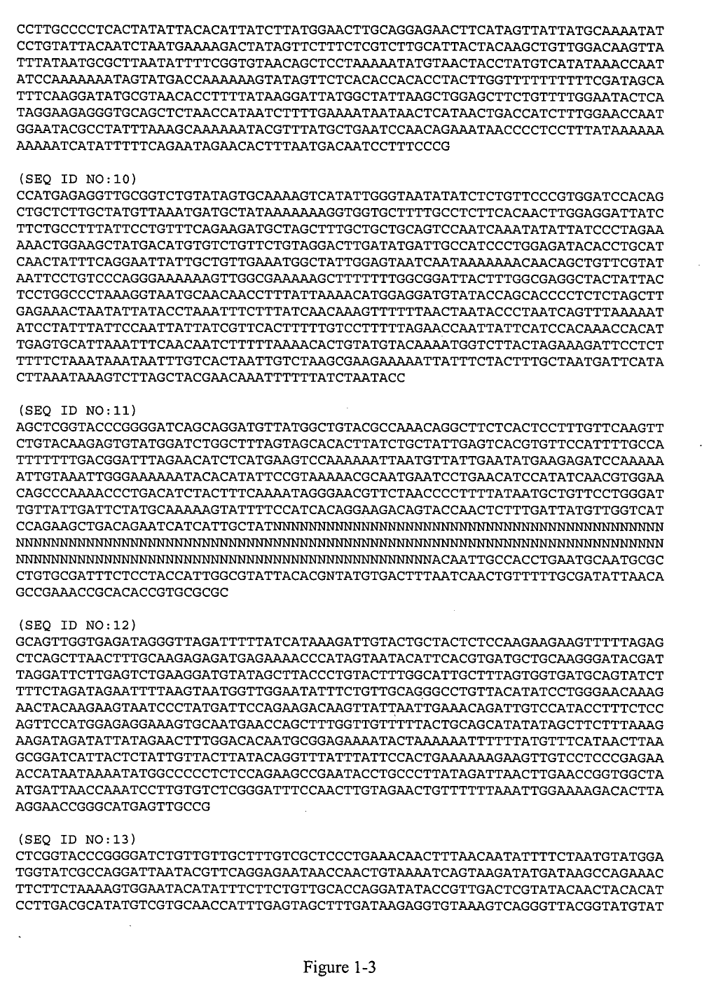

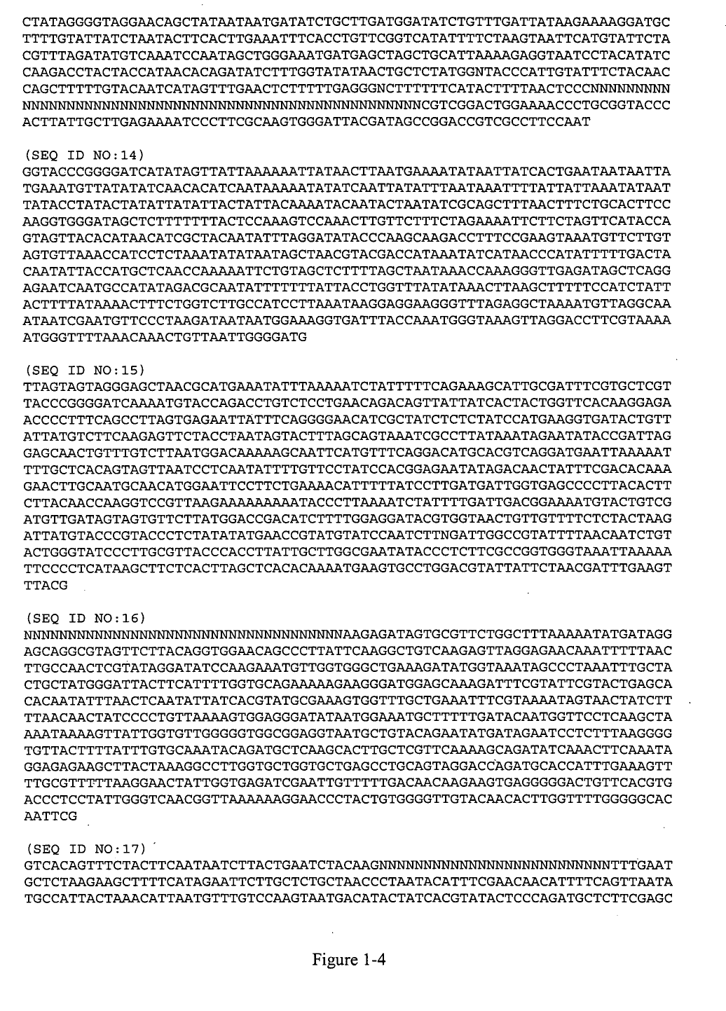

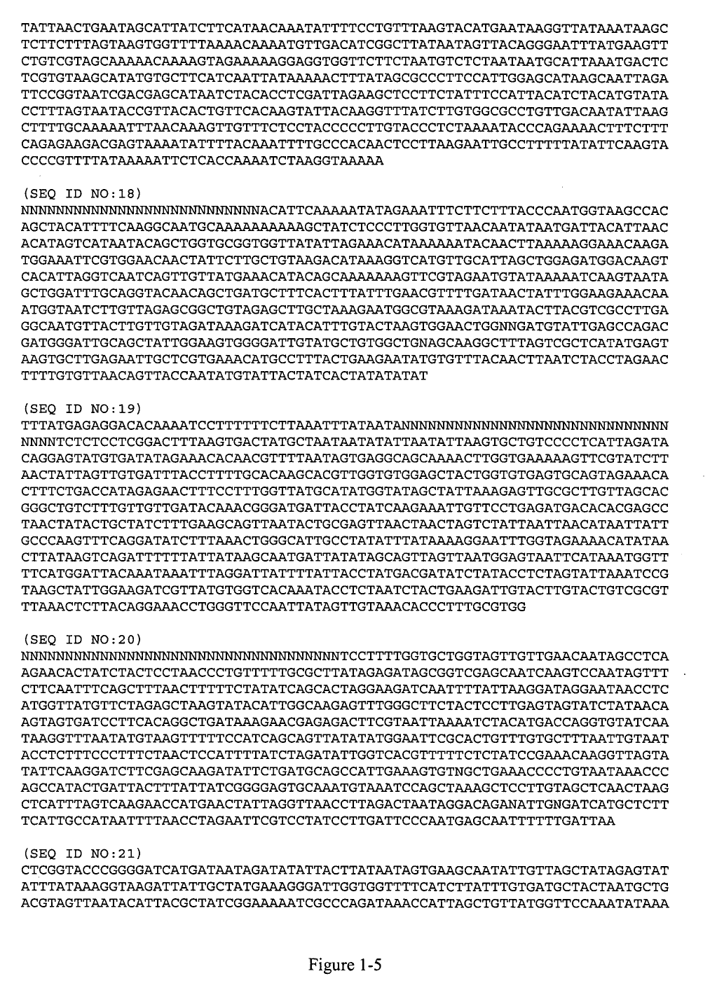

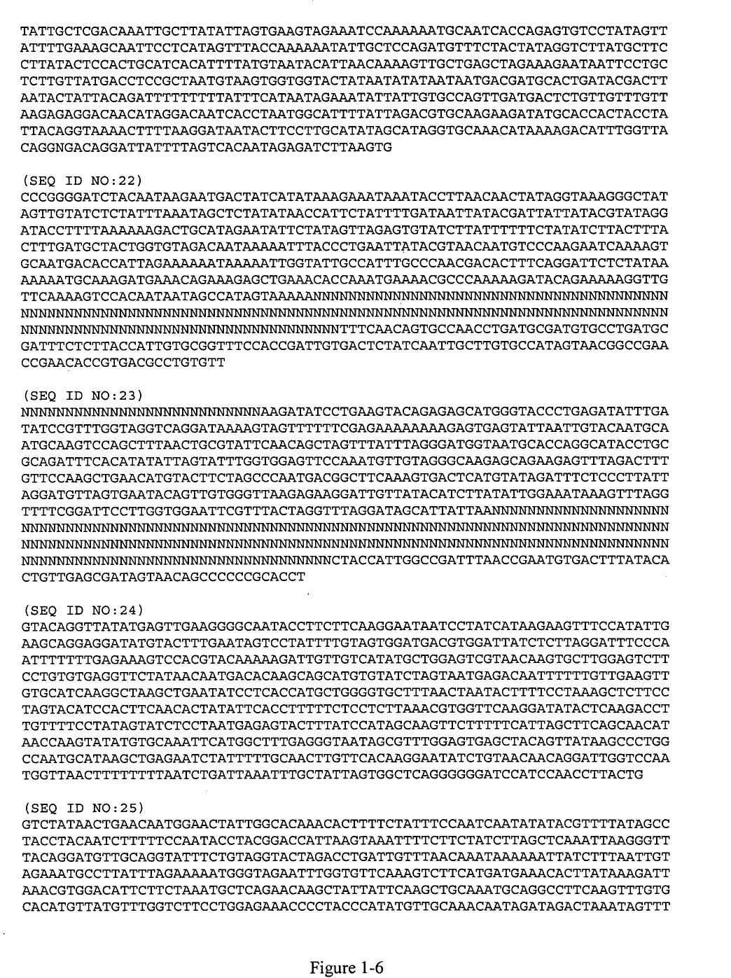

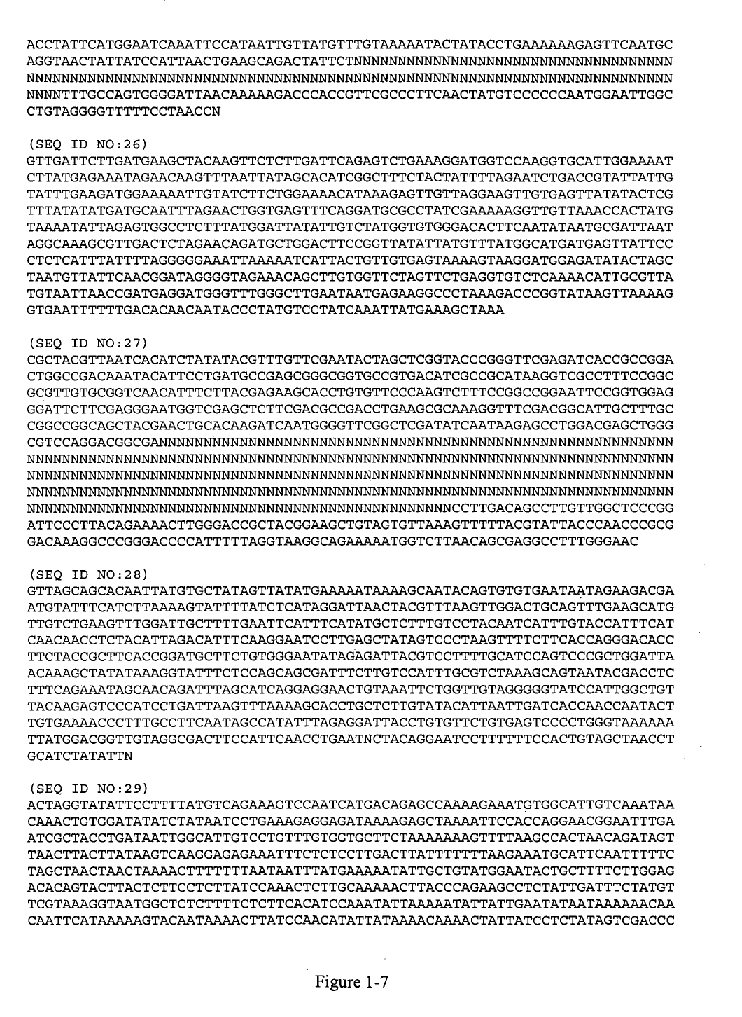

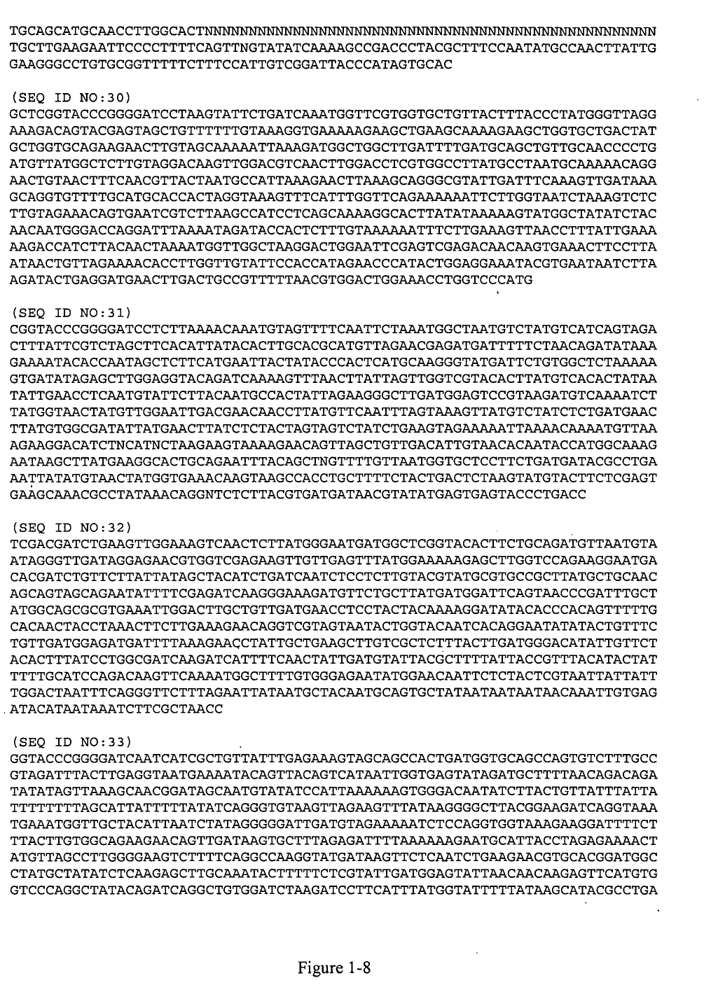

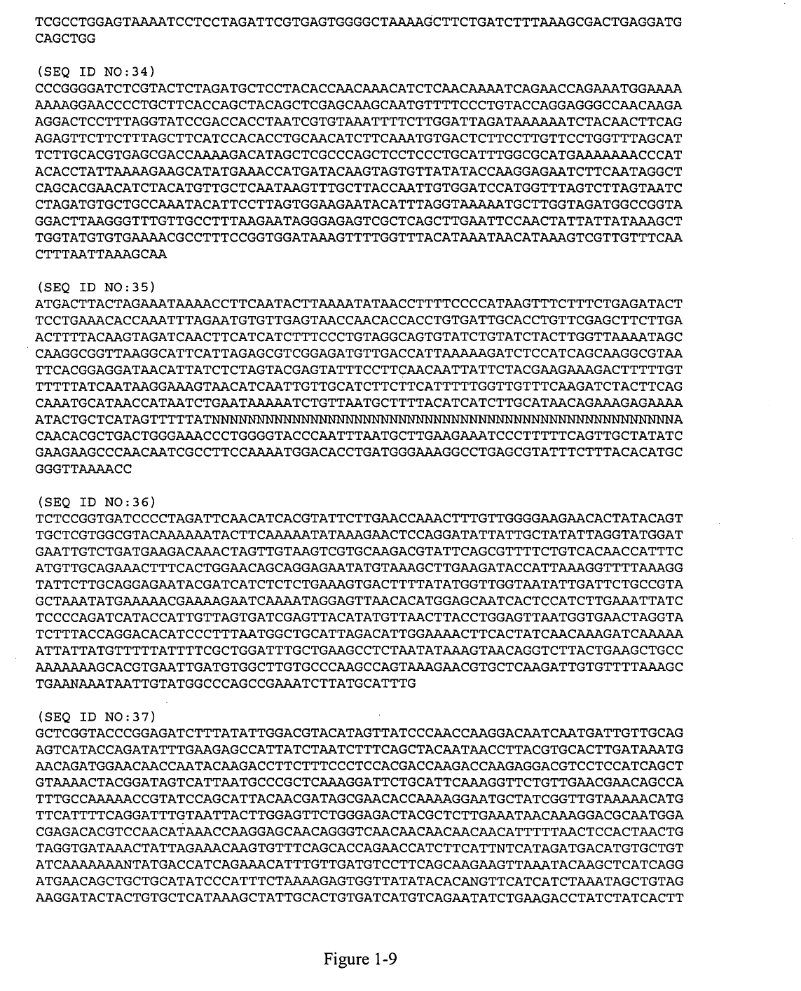

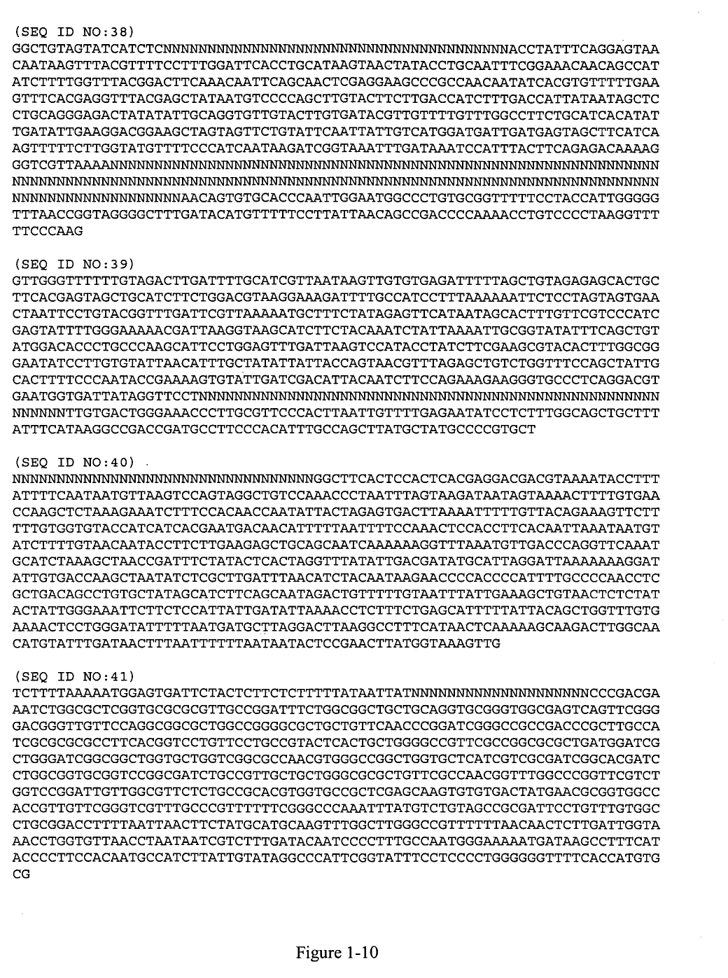

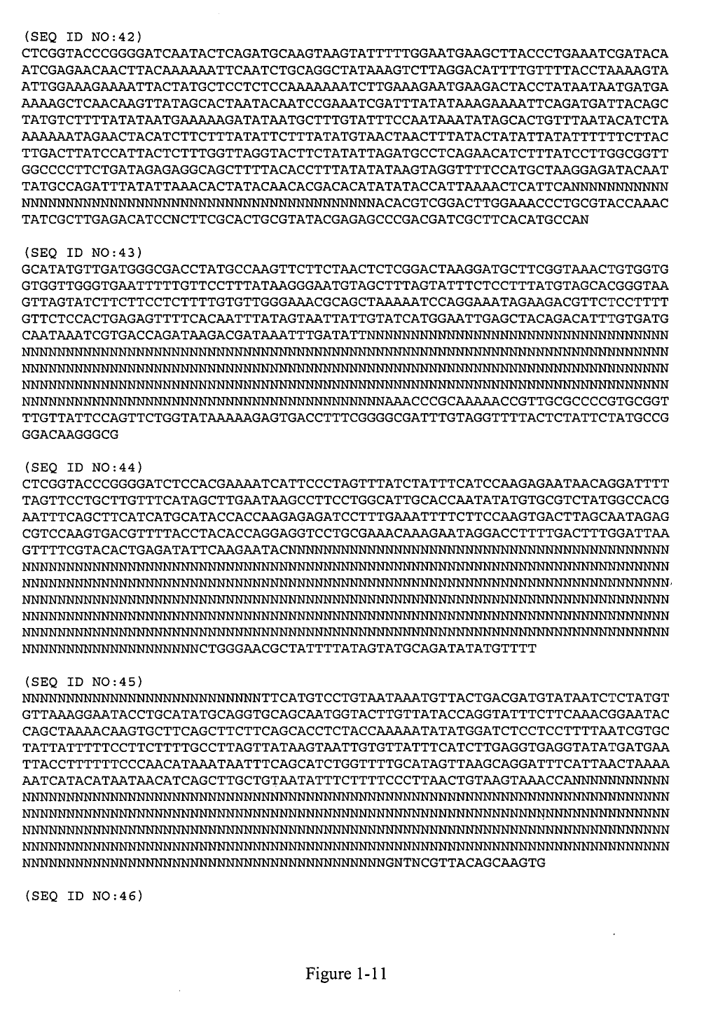

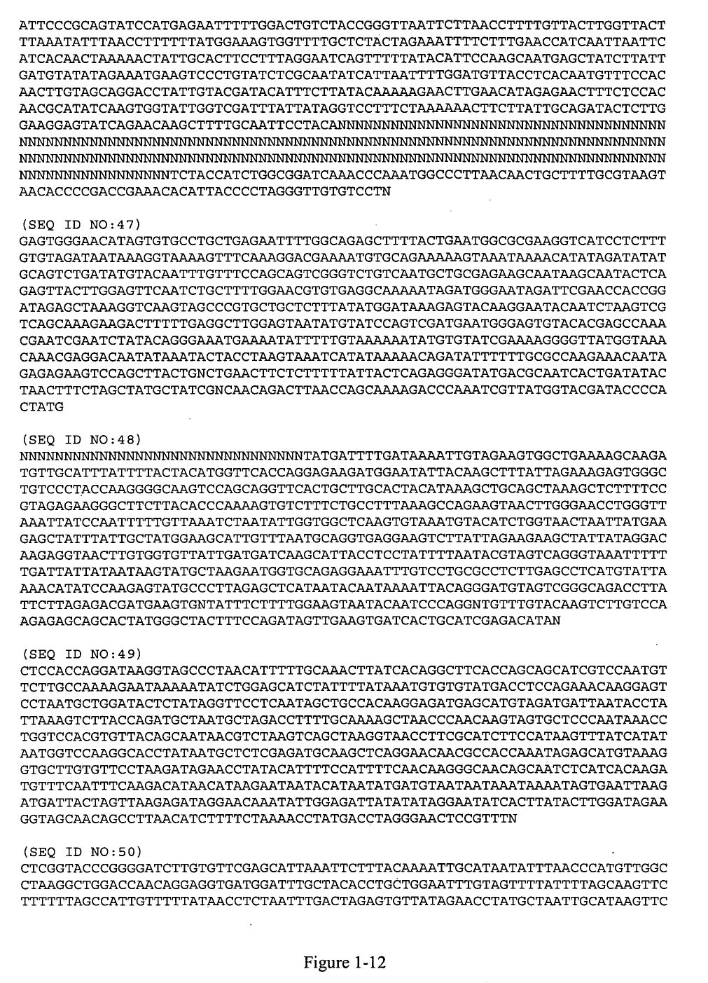

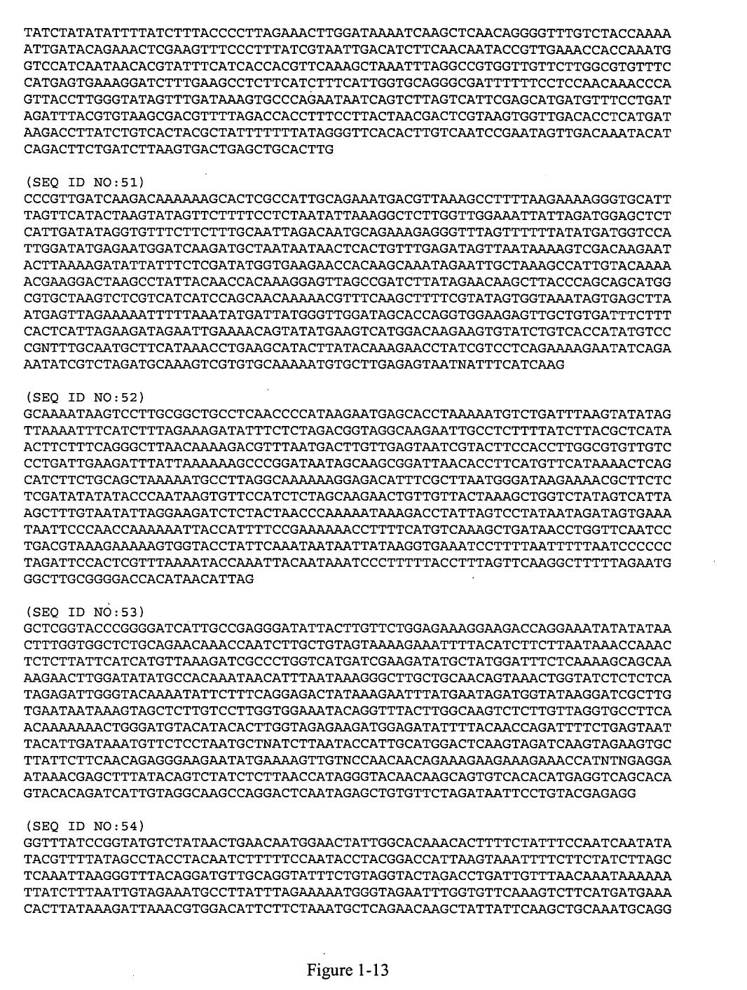

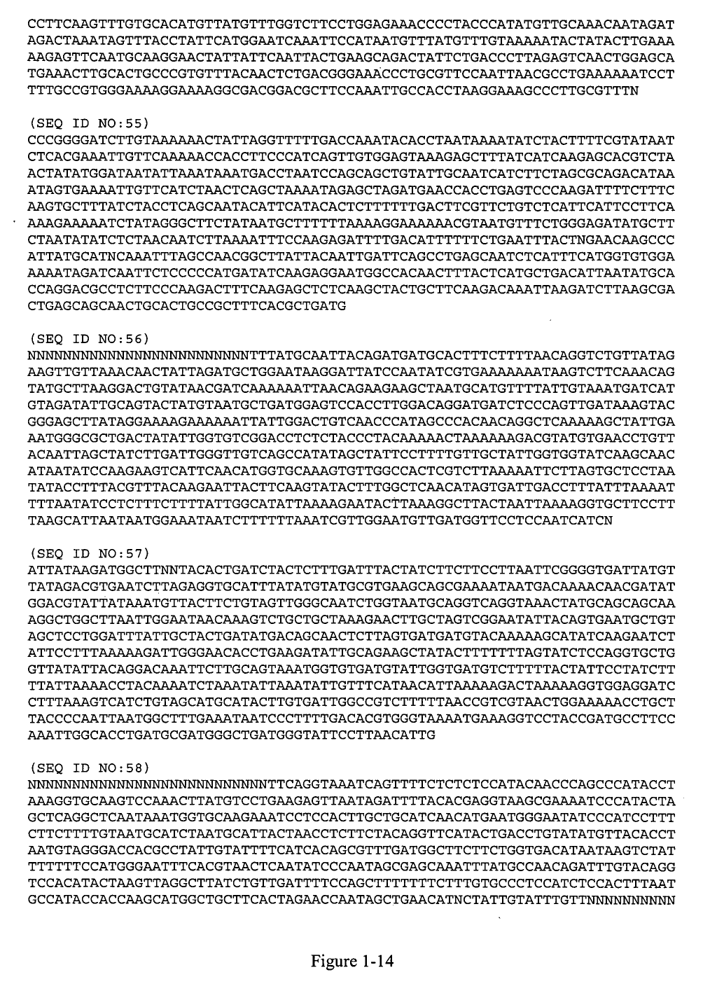

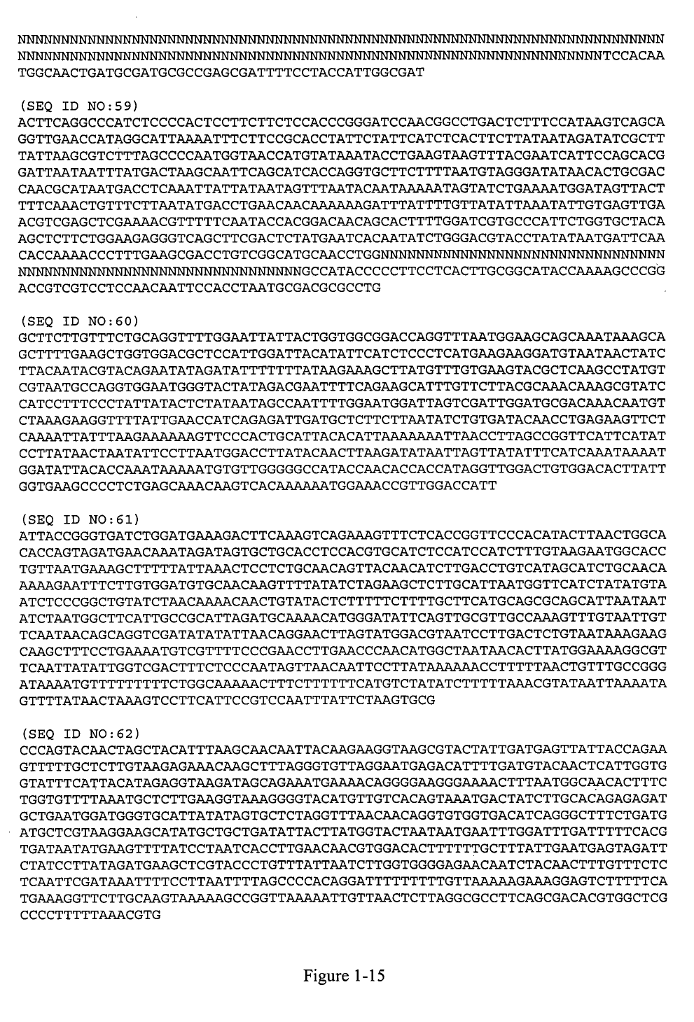

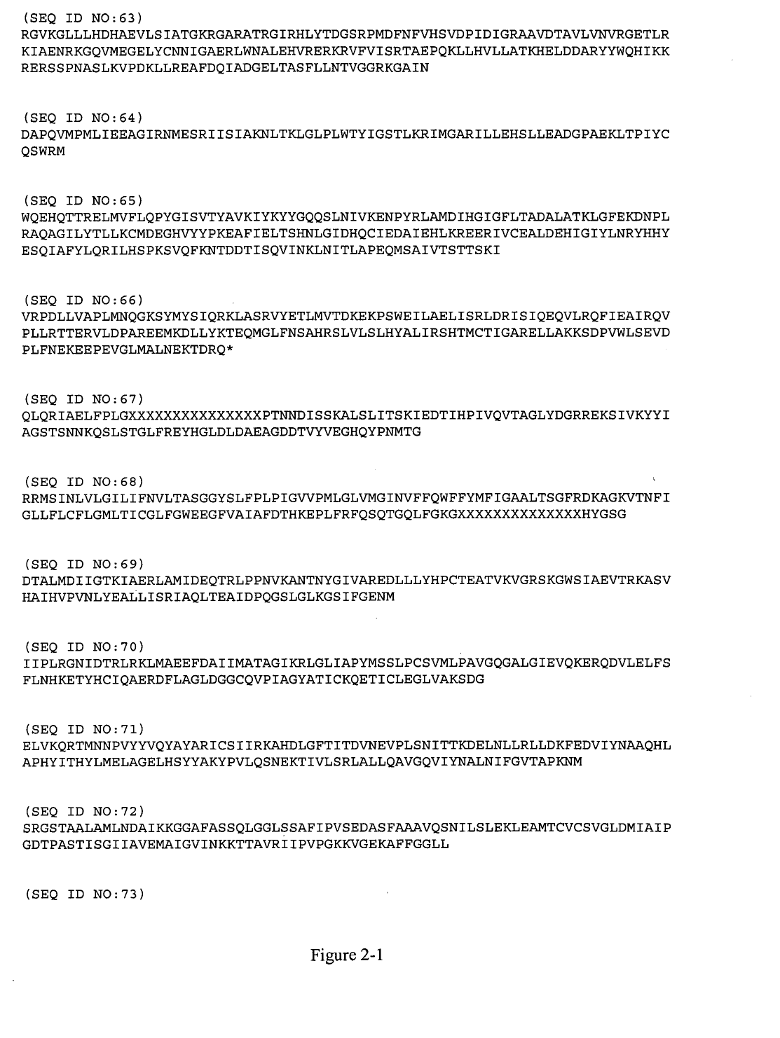

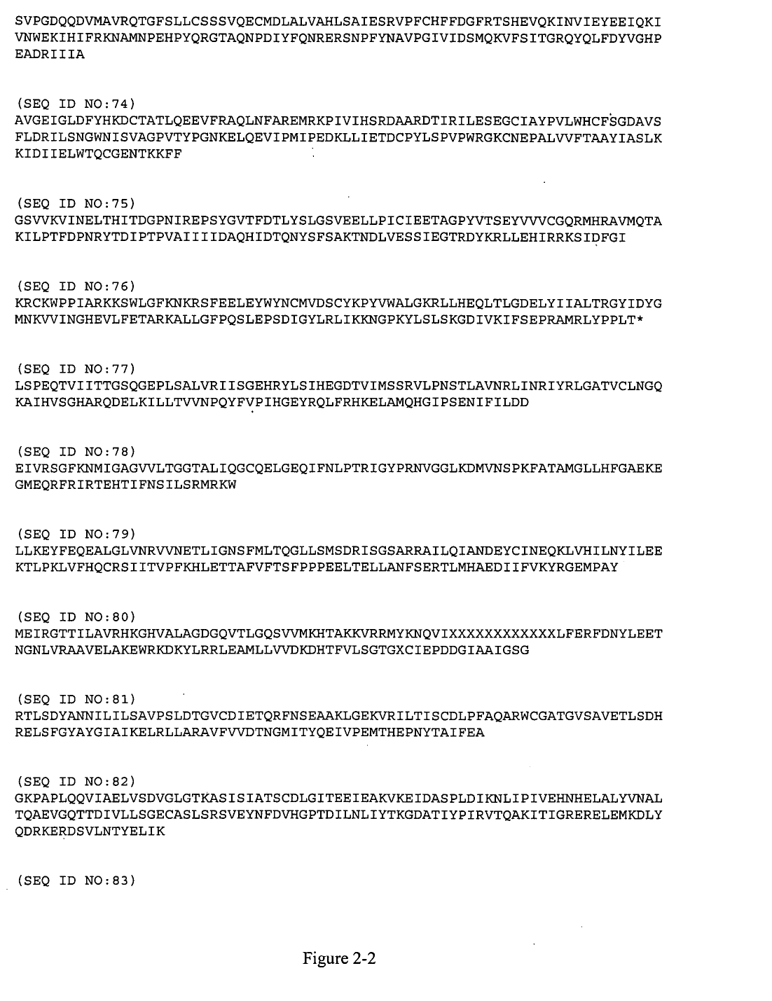

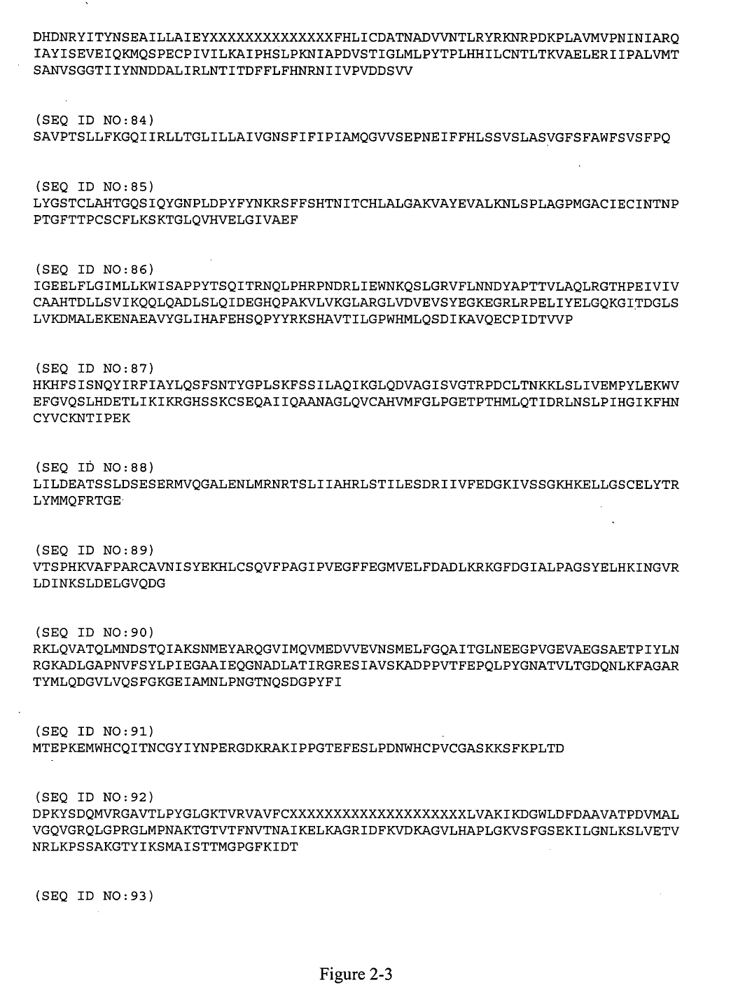

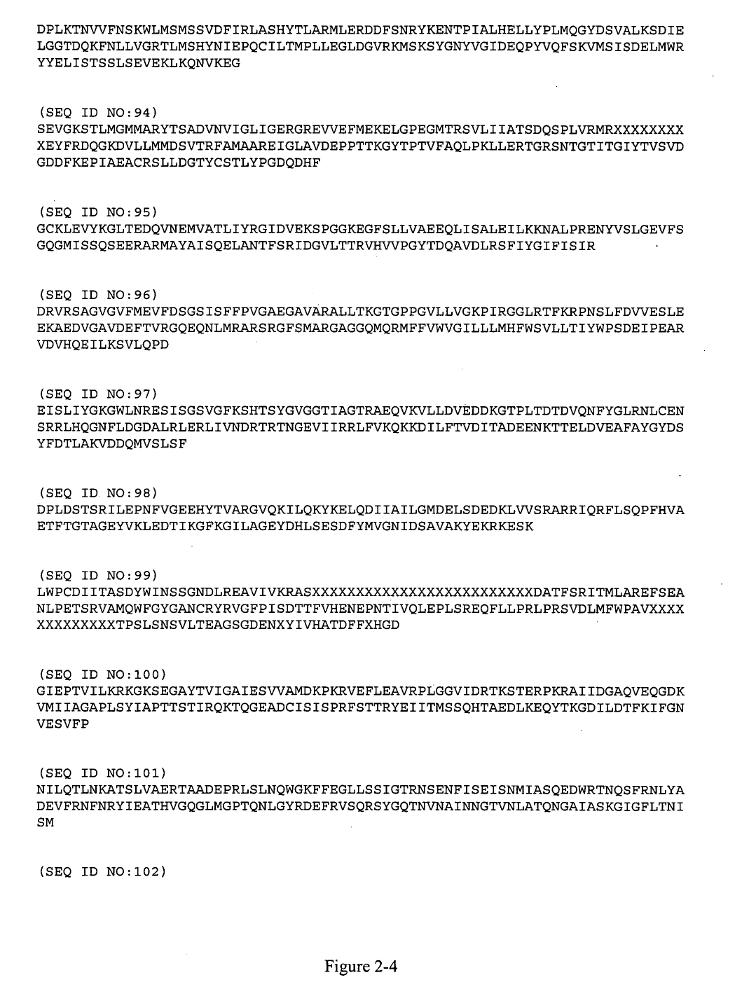

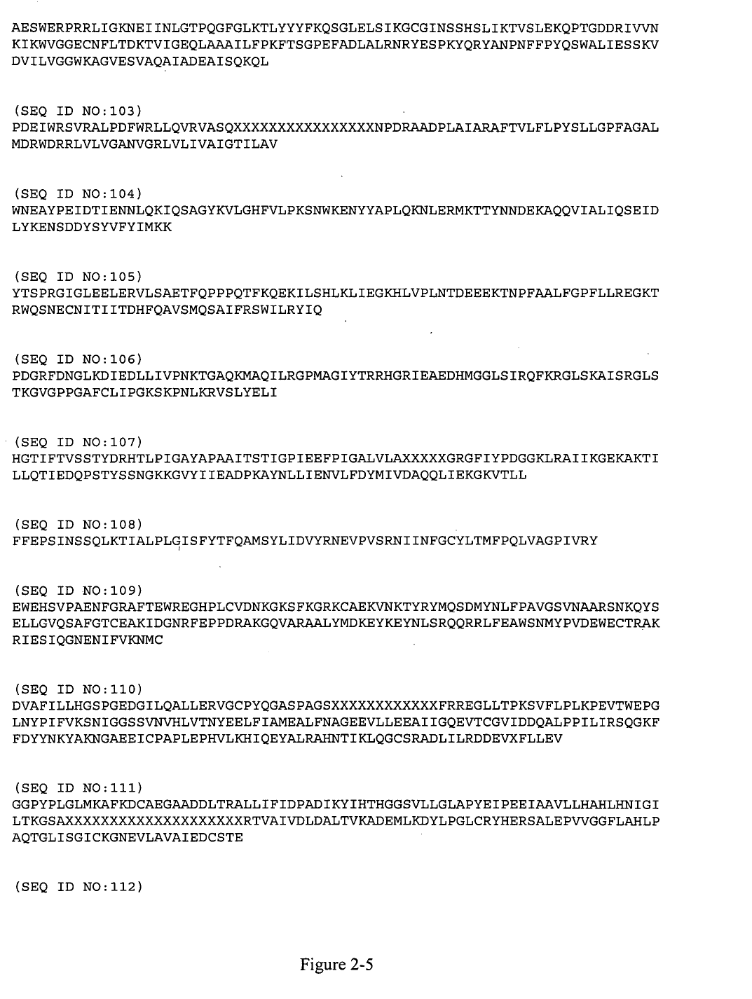

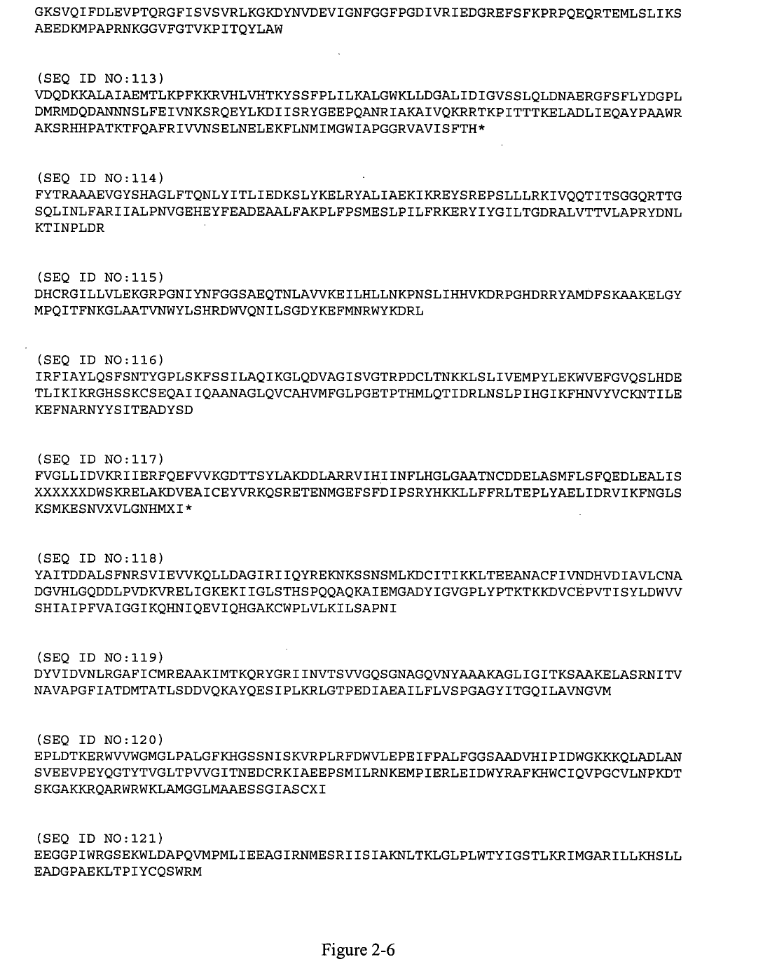

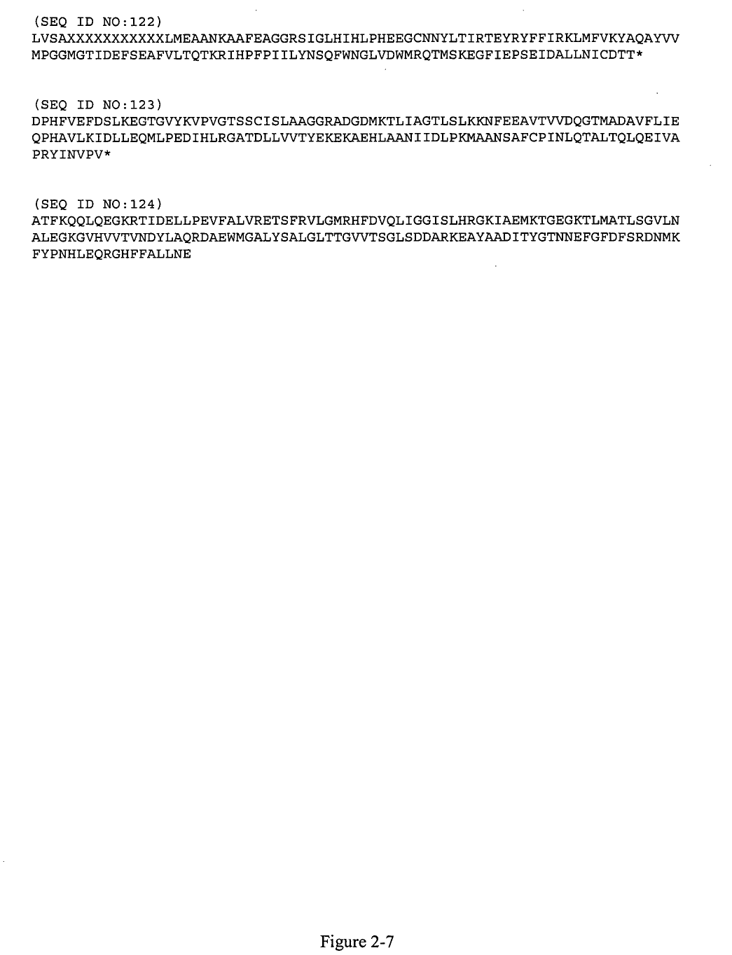

### DETAILED DESCRIPTION

Lawsonia intracellularis, the agent of proliferative enteropathy, is an obligate intracellular pathogen. Very little is known about the genetic basis for the virulence, pathogenesis, or physiology of this bacterium. The present invention provides nucleic acid molecules that are unique to L. intracellularis and therefore, can be used for diagnosis and immunoprophylaxis. The invention also provides the L. intracellularis-specific polypeptides encoded by the nucleic acid molecules of the invention, and antibodies having specific binding affinity for the L. intracellularis-specific polypeptides. The nucleic acid molecules, polypeptides, and antibodies of the invention can be used in methods of the invention to detect L. intracellularis in a sample. The invention additionally provides methods of preventing a L. intracellularis infection in an animal.

Isolated L. intracellularis-Specific Nucleic Acid Molecules

The present invention is based, in part, on the identification of nucleic acid molecules that are unique to L. intracellularis. These nucleic acid molecules are herein referred to as “L. intracellularis-specific” nucleic acid molecules. Particular nucleic acid molecules of the invention include the sequences shown in SEQ ID NOs:1-62 and 131-8727. As used herein, the term “nucleic acid molecule” can include DNA molecules and RNA molecules and analogs of the DNA or RNA molecule generated using nucleotide analogs. A nucleic acid molecule of the invention can be single-stranded or double-stranded, and the strandedness will depend upon its intended use.

The invention further encompasses nucleic acid molecules that differ from the nucleotide sequence of SEQ ID NOs:1-62 and 131-8727. Nucleic acid molecules of the invention include molecules that are at least 10 nucleotides in length and that have at least 75% sequence identity (e.g., at least 80%, 85%, 90%, 95%, or 99% sequence identity) to any of SEQ ID NOs:1-62 and 131-8727. Nucleic acid molecules that differ in sequence from the nucleic acid sequences shown in SEQ ID NOs:1-62 and 131-8727 can be generated by standard techniques, such as site-directed mutagenesis or PCR-mediated mutagenesis. In addition, nucleotide changes can be introduced randomly along all or part of the L. intracellularis-specific nucleic acid molecule, such as by saturation mutagenesis. Alternatively, nucleotide changes can be introduced into a sequence by chemically synthesizing a nucleic acid molecule having such changes.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at http://www.ncbi.nlm.nih.gov. BLAST searches can be performed to determine percent sequence identity between a L. intracellularis-specific nucleic acid molecule of the invention and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence of the invention and another sequence, the default parameters of the respective programs are used. Sequence analysis of the L. intracellularis-specific nucleic acid sequences as performed herein used BLAST version 2.2.3 (updated on Apr. 24, 2002) and 2.2.6 (updated on Apr. 9, 2003).

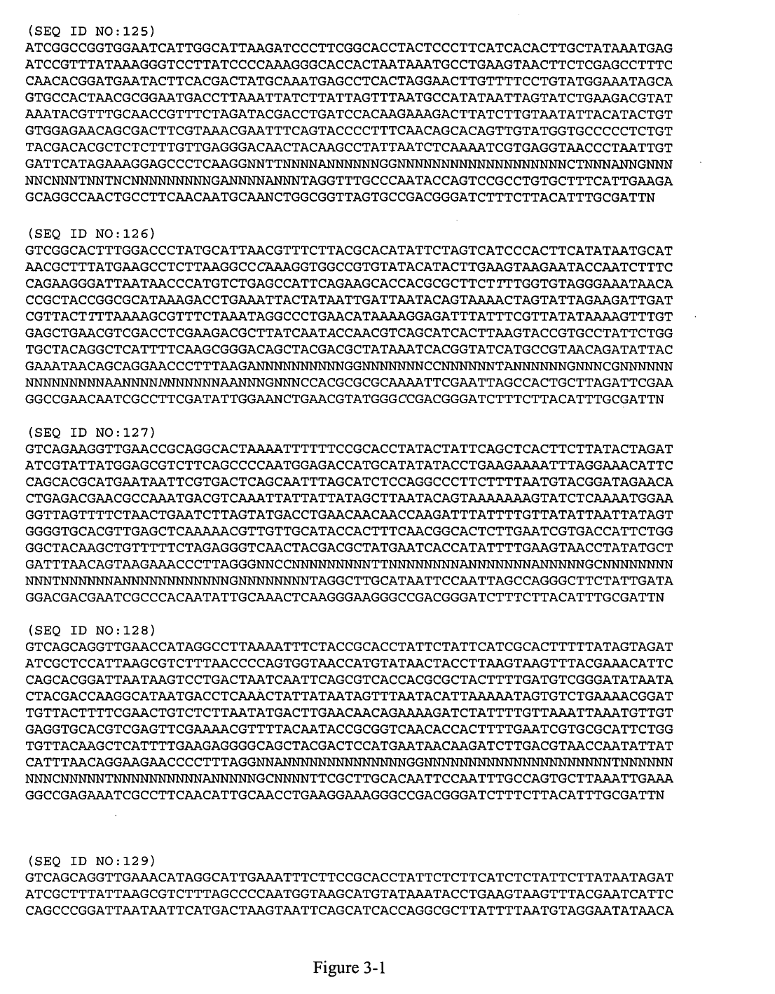

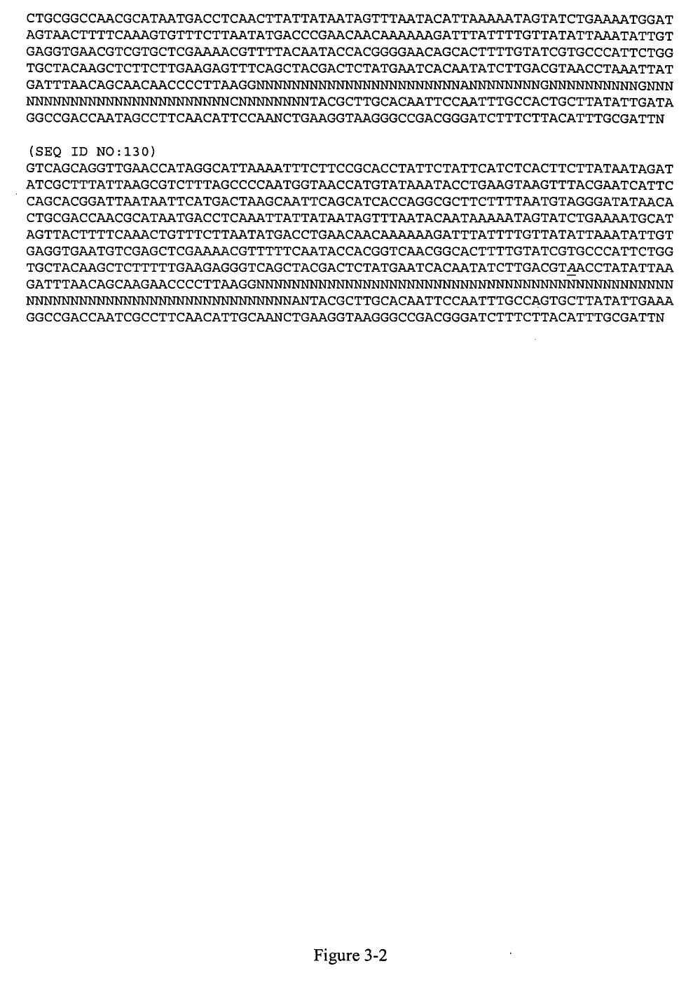

The sequences of representative nucleic acids of the invention having 75%, 80%, 85%, 90%, 95%, and 99% sequence identity to SEQ ID NO:2 are shown in FIG. 3 (SEQ ID NOs:125-130, respectively). Such sequences can be generated using a computer or by hand. The nucleic acid sequences shown in SEQ ID NOs:125-130 were generated by hand by randomly changing 25 nucleotides out of every 100 nucleotides of SEQ ID NO:2, 2 out of every 10, 15 out of every 100, 1 out of every 10, 5 out of every 100, or 1 nucleotide out of every 100 nucleotides of SEQ ID NO:2, respectively. By “changing,” it is meant that the nucleotide at a particular position is replaced randomly with one of the other three nucleotides. It is apparent to those of ordinary skill in the art that any nucleic acid molecule within the scope of the invention can be generated using the same method described herein (i.e., by similarly changing nucleotides within the sequence of SEQ ID NOs:1-62 or 131-8727).

The full-length sizes of representative novel L. intracellularis-specific nucleic acid molecules having the sequences shown in SEQ ID NOs:1-62 are indicated in Table 1.

TABLE 1
Sizes of L. intracellularis-specific
nucleic acids and polypeptides

| GenBank | SEQ ID | Nucleic Acid | | Polypeptide |
|---|---|---|---|---|
| Accession No. | NO: | (bp) | SEQ ID NO: | (amino acids) |
| BH795457 | 1 | 740 | 63 | 192 |
| BH795458 | 2 | 729 | 64 | 78 |
| BH795459 | 3 | 778 | 65 | 200 |
| BH795460 | 4 | 787 | 66 | 169 |
| BH795461 | 5 | 734 | 67 | 118 |
| BH795462 | 6 | 748 | 68 | 141 |
| BH795463 | 7 | 767 | 69 | 115 |
| BH795464 | 8 | 799 | 70 | 125 |
| BH795465 | 9 | 852 | 71 | 136 |
| BH795466 | 10 | 847 | 72 | 121 |
| BH795467 | 11 | 754 | 73 | 154 |
| BH795468 | 12 | 752 | 74 | 165 |
| BH795469 | 13 | 794 | 75 | 142 |
| BH795470 | 14 | 762 | 76 | 144 |
| BH795471 | 15 | 881 | 77 | 131 |
| BH795472 | 16 | 809 | 78 | 98 |
| BH795473 | 17 | 844 | 79 | 141 |
| BH795474 | 18 | 776 | 80 | 131 |
| BH795475 | 19 | 860 | 81 | 126 |
| BH795476 | 20 | 797 | 82 | 163 |
| BH795477 | 21 | 772 | 83 | 189 |
| BH795478 | 22 | 753 | 84 | 72 |
| BH795479 | 23 | 762 | 85 | 103 |
| BH795480 | 24 | 727 | 86 | 207 |
| BH795481 | 25 | 752 | 87 | 157 |
| BH795482 | 26 | 711 | 88 | 83 |
| BH795483 | 27 | 872 | 89 | 88 |
| BH795484 | 28 | 742 | 90 | 181 |
| BH795485 | 29 | 780 | 91 | 60 |
| BH795486 | 30 | 789 | 92 | 176 |
| BH795487 | 31 | 795 | 93 | 169 |
| BH795488 | 32 | 754 | 94 | 178 |
| BH795489 | 33 | 737 | 95 | 136 |
| BH795490 | 34 | 745 | 96 | 161 |
| BH795491 | 35 | 741 | 97 | 163 |
| BH795492 | 36 | 773 | 98 | 129 |
| BH795493 | 37 | 803 | 99 | 187 |
| BH795494 | 38 | 811 | 100 | 152 |
| BH795495 | 39 | 716 | 101 | 148 |
| BH795496 | 40 | 785 | 102 | 175 |
| BH795497 | 41 | 805 | 103 | 103 |
| BH795498 | 42 | 794 | 104 | 91 |
| BH795499 | 43 | 741 | 105 | 108 |
| BG795500 | 44 | 788 | 106 | 103 |
| BH795501 | 45 | 789 | 107 | 131 |
| BH795502 | 46 | 772 | 108 | 66 |
| BH795503 | 47 | 735 | 109 | 163 |
| BH795504 | 48 | 791 | 110 | 208 |
| BH795505 | 49 | 713 | 111 | 172 |
| BH795506 | 50 | 765 | 112 | 101 |
| BH795507 | 51 | 791 | 113 | 196 |
| BH795508 | 52 | 756 | 114 | 154 |
| BH795509 | 53 | 799 | 115 | 117 |
| BH795510 | 54 | 726 | 116 | 164 |
| BH795511 | 55 | 766 | 117 | 163 |
| BH795512 | 56 | 796 | 118 | 187 |
| BH795513 | 57 | 776 | 119 | 138 |
| BH795514 | 58 | 776 | 120 | 179 |
| BH795515 | 59 | 771 | 121 | 92 |
| BH795516 | 60 | 711 | 122 | 141 |
| BH795517 | 61 | 777 | 123 | 154 |
| BH795518 | 62 | 746 | 124 | 164 |

Tables 2, 3, 4, and 5 (contained on the appended compact disc, which has been incorporated by reference herein) represent sequences from L. intracellularis' four genetic elements (plasmids 1, 2, and 3, and the chromosome, respectively), with each consecutive SEQ ID NO corresponding to consecutive 200 bp fragments from the respective genetic element. For example, SEQ ID NO:131 corresponds to nucleotide positions 1 to 200 of plasmid 1 (SEQ ID NO:8736), SEQ ID NO:132 corresponds to nucleotide positions 201 to 400 of plasmid 1 (SEQ ID NO:8736), and so forth. It would be apparent to one of skill in the art that any number of contiguous or non-contiguous fragments from any of the genetic elements of L. intracellularis can be joined together to generate a longer L. intracellularis-specific nucleic acid. Similarly, any number of fragments can be generated, using standard recombinant or synthetic nucleic acid procedures, that span one or more of the fragment junctions represented in Tables 2, 3, 4, and 5.

Using Tables 2, 3, 4, and 5 as references, any nucleic acid molecule of the invention that is between 10 and N nucleotides in length will, under standard amplification conditions, generate an amplification product in the presence of L. intracellularis nucleic acid using an appropriate second nucleic acid molecule (e.g., an oligonucleotide primer) but will not generate an amplification product from nucleic acid of any of the organisms shown in Tables 2, 3, 4, or 5 corresponding to the respective SEQ ID NO, using an appropriate third nucleic acid molecule (e.g., an oligonucleotide primer that specifically anneals to nucleic acid from the other organism). For example, for SEQ ID NO:132 (fragment 2 of plasmid 1), any such molecule that is 10 to 21 nucleotides in length, under standard amplification conditions, generates an amplification product from L. intracellularis nucleic acid using an appropriate second nucleic acid molecule, but does not generate an amplification product from nucleic acid of Homo sapiens or Danio rerio using an appropriate third nucleic acid molecule.

With respect to the organisms identified in Tables 2, 3, 4, and 5, some of them represent multiple species, subspecies, or strains. To test whether or not particular reagents distinguish between L. intracellularis and such species, subspecies, or strains, it may be desirable to test a representative number of species, subspecies, or strains, respectively. In cases where the genetic variation is minimal within the species, subspecies, or strains, it may not be necessary to test more than one or two species, subspecies, or strains, respectively. In other cases, multiple species, subspecies, or strains may need to be tested, although initial testing can focus on the most genetically distant species, subspecies, or strains, respectively.

As used herein, “standard amplification conditions” refer to the basic components of an amplification reaction mix, and cycling conditions that include multiple cycles of denaturing the template nucleic acid, annealing the oligonucleotide primers to the template nucleic acid, and extension of the primers by the polymerase to produce an amplification product (see, for example, U.S. Pat. Nos. 4,683,195; 4,683,202; 4,800,159; and 4,965,188). The basic components of an amplification reaction mix generally include, for example, about 10-25 nmole of each of the four deoxynucleoside triphosphates, (e.g., dATP, dCTP, dTTP, and dGTP, or analogs thereof), 10-100 pmol of primers, template nucleic acid, and a polymerase enzyme. The reaction components are generally suspended in a buffered aqueous solution having a pH of between about 7 and about 9. The aqueous buffer can further include one or more co-factors (e.g., Mg²⁺, K⁺) required by the polymerase. Additional components such as DMSO are optional. Template nucleic acid is typically denatured at a temperature of at least about 90° C., and extension from primers is typically performed at a temperature of at least about 72° C.

The annealing temperature can be used to control the specificity of amplification. The temperature at which primers anneal to template nucleic acid must be below the Tm of each of the primers, but high enough to avoid non-specific annealing of primers to the template nucleic acid. The Tm is the temperature at which half of the DNA duplexes have separated into single strands, and can be predicted for an oligonucleotide primer using the formula provided in section 11.46 of Sambrook et al. (1989, Molecular Cloning: A Laboratory Manual, 2nd Ed., Cold Spring Harbor Laboratory Press, Cold Spring Harbor, N.Y.). Non-specific amplification products are detected as bands on a gel that are not the size expected for the correct amplification product. The annealing temperature used in amplification reactions to demonstrate that the claimed nucleic acid molecules are L. intracellularis-specific can be 57° C. It can be appreciated by those of skill in the art that appropriate positive and negative controls should be performed with every set of amplification reactions to avoid uncertainties related to contamination and/or non-specific annealing of oligonucleotide primers and extension therefrom.

An appropriate second nucleic acid molecule is generally an oligonucleotide primer that specifically anneals to L. intracellularis nucleic acid and that can act in combination with a nucleic acid molecule of the invention, specifically, for example, a 10 to 30-, or 40-, or 50-nucleotide-long nucleic acid molecule of the invention, under appropriate amplification conditions to generate an amplification product in the presence of L. intracellularis nucleic acid. In order for a second nucleic acid molecule to act in combination with a nucleic acid molecule of the invention to generate an amplification product, the two molecules must anneal to opposite strands of the template nucleic acid, and should be an appropriate distance from one another such that the polymerase can effectively polymerize across the region and such that the amplification product can be readily detected using, for example, electrophoresis. Oligonucleotide primers can be designed using, for example, a computer program such as OLIGO (Molecular Biology Insights Inc., Cascade, Colo.) to assist in designing primers that have similar melting temperatures. Typically, oligonucleotide primers can be 10 to 50 nucleotides in length (e.g., 12, 14, 16, 18, 20, 22, 24, 26, 28, 30, 32, 34, 36, 38, 40, 42, 44, 46, 48, or 50 nucleotides in length).

Representative pairs of oligonucleotide primers that were used to amplify each of the L. intracellularis-specific nucleic acid molecules of the invention are shown in Table 8 (SEQ ID NOs:8728-8735). Alternatively, the nucleic acid molecules having the sequences shown in SEQ ID NOs:1-62 and 131-8727 can be used to design a pair of oligonucleotide primers. Oligonucleotides of the invention can be obtained by restriction enzyme digestion of L. intracellularis-specific nucleic acid molecules or can be prepared by standard chemical synthesis and other known techniques.

As used herein, an organism other than L. intracellularis refers to any organism that is not L. intracellularis. Generally, only relevant organisms are used in amplification reactions to examine the specificity of a 10 or more nucleotide-long nucleic acid molecule of the invention. Particularly relevant organisms include, without limitation, Brachyspira hyodysenteria, Brachyspira pylosicoli, E. coli, Salmonella typhimurium, Salmonella choleraesuis, Bilophila wadsworthiae, and Clostridium difficile.

As used herein, an “isolated” nucleic acid molecule is a nucleic acid molecule that is separated from other nucleic acid molecules that are usually associated with the isolated nucleic acid molecule. Thus, an “isolated” nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamine) containing restriction-digested genomic DNA is not to be considered an isolated nucleic acid.

Isolated nucleic acid molecules of the invention can be obtained using techniques routine in the art. For example, isolated nucleic acids within the scope of the invention can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid molecule of the invention. Isolated nucleic acids of the invention also can be chemically synthesized, either as a single nucleic acid molecule or as a series of oligonucleotides. In addition, isolated nucleic acid molecules of the invention also can be obtained by mutagenesis. For example, an isolated nucleic acid that shares identity with an art known sequence can be mutated using common molecular cloning techniques (e.g., site-directed mutagenesis). Possible mutations include, without limitation, deletions, insertions, substitutions, and combinations thereof.

Vectors containing L. intracellularis-specific nucleic acid molecules also are provided by the invention. Vectors, including expression vectors, suitable for use in the present invention are commercially available and/or produced by recombinant DNA technology methods routine in the art. A vector containing a L. intracellularis-specific nucleic acid molecule can have elements necessary for expression operably linked to such a L. intracellularis-specific nucleic acid, and further can include sequences such as those encoding a selectable marker (e.g., an antibiotic resistance gene), and/or those that can be used in purification of a L. intracellularis-specific polypeptide (e.g., 6×His tag).

Elements necessary for expression include nucleic acid sequences that direct and regulate expression of nucleic acid coding sequences. One example of an element necessary for expression is a promoter sequence, for example, a L. intracellularis-specific promoter (e.g., from the same coding sequence being expressed or from a different coding sequence) or a non-L. intracellularis-specific promoter. Elements necessary for expression also can include introns, enhancer sequences, response elements, or inducible elements that modulate expression of a L. intracellularis-specific nucleic acid. Elements necessary for expression can be of bacterial, yeast, insect, mammalian, or viral origin and vectors can contain a combination of elements from different origins. Elements necessary for expression are described, for example, in Goeddel, 1990, Gene Expression Technology: Methods in Enzymology, 185, Academic Press, San Diego, Calif. As used herein, operably linked means that a promoter and/or other regulatory element(s) are positioned in a vector relative to a L. intracellularis-specific nucleic acid in such a way as to direct or regulate expression of the L. intracellularis-specific nucleic acid. Many methods for introducing nucleic acids into cells, both in vivo and in vitro, are well known to those skilled in the art and include, without limitation, calcium phosphate precipitation, electroporation, heat shock, lipofection, microinjection, and viral-mediated nucleic acid transfer.

Another aspect of the invention pertains to host cells into which a vector of the invention, e.g., an expression vector, or an isolated nucleic acid molecule of the invention has been introduced. The term “host cell” refers not only to the particular cell but also to the progeny or potential progeny of such a cell. A host cell can be any prokaryotic or eukaryotic cell. For example, L. intracellularis-specific nucleic acids can be expressed in bacterial cells such as E. coli, or in insect cells, yeast or mammalian cells (such as Chinese hamster ovary cells (CHO) or COS cells). Other suitable host cells are known to those skilled in the art.

Vectors containing nucleic acid molecules unique to L. intracellularis were deposited with the American Type Culture Collection (ATCC), 10801 University Boulevard Manassas, Va. 20110, on ______, and assigned Accession Numbers ______, ______, ______, and ______. Each deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

Purified L. intracellularis Polypeptides

One aspect of the invention pertains to purified L. intracellularis-specific polypeptides, as well as polypeptide fragments. A “L. intracellularis-specific polypeptide” refers to a polypeptide encoded by a nucleic acid molecule that is unique to L. intracellularis (e.g., L. intracellularis-specific nucleic acid molecules, for example, those having the sequences shown in SEQ ID NOs:1-62 and 131-8727). Predicted amino acid sequences encoded by L. intracellularis-specific nucleic acids of the invention are shown in SEQ ID NOs:63-124.

The term “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

L. intracellularis-specific polypeptides can be purified from natural sources (e.g., a biological sample) by known methods such as DEAE ion exchange, gel filtration, and hydroxyapatite chromatography. A purified L. intracellularis-specific polypeptide also can be obtained by expressing a L. intracellularis-specific nucleic acid in an expression vector, for example. In addition, a purified L. intracellularis-specific polypeptide can be obtained by chemical synthesis. The extent of purity of a L. intracellularis-specific polypeptide can be measured using any appropriate method, e.g., column chromatography, polyacrylamide gel electrophoresis, or HPLC analysis.

In addition to naturally-occurring L. intracellularis-specific polypeptides, the skilled artisan will further appreciate that changes can be introduced into a nucleic acid molecule (e.g., those having the sequence shown in SEQ ID NOs:1-62 and 131-8727) as discussed herein, thereby leading to changes in the amino acid sequence of the encoded polypeptide. For example, changes can be introduced into L. intracellularis-specific nucleic acid coding sequences leading to conservative and/or non-conservative amino acid substitutions at one or more amino acid residues. A “conservative amino acid substitution” is one in which one amino acid residue is replaced with a different amino acid residue having a similar side chain. Similarity between amino acid residues has been assessed in the art. For example, Dayhoff et al. (1978, in Atlas of Protein Sequence and Structure, Vol. 5, Suppl. 3, pp 345-352) provides frequency tables for amino acid substitutions that can be employed as a measure of amino acid similarity. A non-conservative substitution is one in which an amino acid residue is replaced with an amino acid residue that does not have a similar side chain.

The invention also provides for chimeric or fusion polypeptides. As used herein, a “chimeric” or “fusion” polypeptide includes a L. intracellularis-specific polypeptide operatively linked to a heterologous polypeptide. A heterologous polypeptide can be at either the N-terminus or C-terminus of the L. intracellularis-specific polypeptide. Within a chimeric or fusion polypeptide, the term “operatively linked” is intended to indicate that the two polypeptides are encoded in-frame relative to one another. In a fusion polypeptide, the heterologous polypeptide generally has a desired property such as the ability to purify the fusion polypeptide (e.g., by affinity purification). A chimeric or fusion polypeptide of the invention can be produced by standard recombinant DNA techniques, and can use commercially available vectors.

A polypeptide commonly used in a fusion polypeptide for purification is glutathione S-transferase (GST), although numerous other polypeptides are available and can be used. In addition, a proteolytic cleavage site can be introduced at the junction between a L. intracellularis-specific polypeptide and a non-L. intracellularis-specific polypeptide to enable separation of the two polypeptides subsequent to purification of the fusion polypeptide. Enzymes that cleave such proteolytic sites include Factor Xa, thrombin, or enterokinase. Representative expression vectors encoding a heterologous polypeptide that can be used in affinity purification of a L. intracellularis polypeptide include pGEX (Pharmacia Biotech Inc; Smith & Johnson, 1988, Gene, 67:31-40), pMAL (New England Biolabs, Beverly, Mass.) and pRIT5 (Pharmacia, Piscataway, N.J.).

Anti-L. intracellularis-Specific Antibodies

Another aspect of the invention relates to anti-L. intracellularis-specific antibodies. The term “anti-L. intracellularis-specific antibodies” as used herein refers to immunoglobulin molecules and immunologically active portions of immunoglobulin molecules that have specific binding affinity for a L. intracellularis-specific polypeptide. The invention provides polyclonal and monoclonal antibodies that have specific binding affinity for L. intracellularis-specific polypeptides. The sequences of numerous L. intracellularis-specific polypeptides that can be used to generate anti-L. intracellularis-specific antibodies are disclosed herein (e.g., SEQ ID NOs:63-124). Examples of immunologically active portions of immunoglobulin molecules include F(ab) and F(ab′)₂ fragments, which can be generated by treating an immunoglobulin molecule with an enzyme such as pepsin. As used herein, an antibody that has “specific binding affinity” for a L. intracellularis-specific polypeptide is an antibody that binds a L. intracellularis-specific polypeptide but does not bind a non-L. intracellularis-specific polypeptides. A non-L. intracellularis-specific polypeptide as used herein refers to a polypeptide that may or may not be found in L. intracellularis, but is found in at least one other organism besides L. intracellularis.

A purified L. intracellularis-specific polypeptide or a fragment thereof can be used as an immunogen to generate polyclonal or monoclonal antibodies that have specific binding affinity for L. intracellularis-specific polypeptides. Such antibodies can be generated using standard techniques as described herein. Full-length L. intracellularis-specific polypeptides (see Table 1) or, alternatively, antigenic fragments of L. intracellularis-specific polypeptides can be used as immunogens. An antigenic fragment of a L. intracellularis-specific polypeptide usually includes at least 8 (e.g., 10, 15, 20, or 30) amino acid residues of a L. intracellularis-specific polypeptide (e.g., having the sequence shown in SEQ ID NOs:63-124), and encompasses an epitope of a L. intracellularis-specific polypeptide such that an antibody (e.g., polyclonal or monoclonal) raised against the antigenic fragment has specific binding affinity for a L. intracellularis-specific polypeptide.

Antibodies are typically prepared by first immunizing a suitable animal (e.g., a rabbit, a goat, a mouse or another mammal) with an immunogenic preparation. An appropriate immunogenic preparation can contain, for example, a recombinantly expressed or chemically synthesized L. intracellularis-specific polypeptide, of a fragment thereof. The preparation can further include an adjuvant, such as Freund's complete or incomplete adjuvant, or similar immunostimulatory agent. Immunization of a suitable animal with an immunogenic L. intracellularis-specific polypeptide preparation induces a polyclonal anti-L. intracellularis-specific antibody response.

The titer of the anti-L. intracellularis-specific antibody in the immunized animal can be monitored over time by standard techniques, such as with an enzyme-linked immunosorbent assay (ELISA) using immobilized L. intracellularis-specific polypeptides. If desired, the antibody molecules directed against L. intracellularis-specific polypeptides can be isolated from the animal (e.g., from the blood) and further purified by well-known techniques such as protein A chromatography to obtain the IgG fraction.

At an appropriate time after immunization, e.g., when the anti-L. intracellularis-specific antibody titers are highest, antibody-producing cells can be obtained from the animal and used to prepare monoclonal antibodies by standard techniques, such as the hybridoma technique originally described by Kohler & Milstein (1975, Nature, 256:495-497), the human B cell hybridoma technique (Kozbor et al., 1983, Immunol. Today, 4:72), or the EBV-hybridoma technique (Cole et al., 1985, Monoclonal Antibodies and Cancer Therapy, Alan R. Liss, Inc., pp. 77-96). The technology for producing various monoclonal antibody hybridomas is well known (see, generally, Current Protocols in Immunology, 1994, Coligan et al. (Eds.), John Wiley & Sons, Inc., New York, N.Y.). Briefly, an immortal cell line (e.g., a myeloma cell line) is fused to lymphocytes (e.g., splenocytes) from an animal immunized with an immunogenic L. intracellularis-specific polypeptide as described above, and the culture supernatants of the resulting hybridoma cells are screened to identify a hybridoma producing a monoclonal antibody that has specific binding affinity for the L. intracellularis-specific polypeptide.

Any of the well-known protocols used for fusing lymphocytes and immortalized cell lines can be applied for the purpose of generating an anti-L. intracellularis-specific monoclonal antibody (see, e.g., Current Protocols in Immunology, supra; Galfre et al., 1977, Nature, 266:55052; R. H. Kenneth, in Monoclonal Antibodies: A New Dimension In Biological Analyses, Plenum Publishing Corp., New York, N.Y., 1980; and Lerner, 1981, Yale J. Biol. Med., 54:387-402). Moreover, the ordinary skilled worker will appreciate that there are many variations of such methods that also would be useful. Typically, the immortal cell line is derived from the same species as the lymphocytes. For example, murine hybridomas can be made by fusing lymphocytes from a mouse immunized with an immunogenic preparation with an immortalized mouse cell line, e.g., a myeloma cell line that is sensitive to culture medium containing hypoxanthine, aminopterin and thymidine (“HAT medium”). Any of a number of ATCC-available myeloma cell lines can be used as a fusion partner according to standard techniques, e.g., the P3-NS1/1-Ag4-1, P3-x63-Ag8.653 or Sp2/O—Ag14 myeloma lines. Typically, HAT-sensitive mouse myeloma cells are fused to mouse splenocytes using polyethylene glycol (PEG). Hybridoma cells resulting from the fusion are then selected using HAT medium. Hybridoma cells producing a monoclonal antibody are detected by screening the hybridoma culture supernatants for antibodies that bind L. intracellularis-specific polypeptides, e.g., using a standard ELISA assay.

As an alternative to preparing monoclonal antibody-secreting hybridomas, an anti-L. intracellularis-specific monoclonal antibody can be identified and isolated by screening a recombinant combinatorial immunoglobulin library (e.g., an antibody phage display library) with L. intracellularis-specific polypeptides. Immunoglobulin library members that have specific binding affinity for L. intracellularis-specific polypeptides can be isolated from such libraries. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/20791; PCT Publication No. WO 93/01288; Hay et al., 1992, Hum. Antibod. Hybridomas, 3:81-85; Griffiths et al., 1993, EMBO J, 12:725-734; and references therein.

Additionally, recombinant anti-L. intracellularis-specific antibodies, such as chimeric and humanized monoclonal antibodies, comprising both human and non-human portions, are within the scope of the invention. Such chimeric and humanized monoclonal antibodies can be produced by recombinant DNA techniques known in the art, for example using methods described in PCT Publication No. WO 87/02671; European Patent (EP) Application 184,187; U.S. Pat. No. 4,816,567; Better et al., 1988, Science, 240:1041-1043; Shaw et al., 1988, J. Natl. Cancer Inst., 80:1553-1559); U.S. Pat. No. 5,225,539; Verhoeyan et al., 1988, Science, 239:1534; Beidler et al., 1988, J. Immunol., 141:4053-4060; and references therein.

An anti-L. intracellularis-specific antibody (e.g., a monoclonal antibody) can be used to isolate L. intracellularis-specific polypeptides by standard techniques, such as affinity chromatography or immunoprecipitation. An anti-L. intracellularis-specific antibody can facilitate the purification of natural L. intracellularis-specific polypeptides from cells and of recombinantly-produced L. intracellularis-specific polypeptides expressed in host cells. Moreover, an anti-L. intracellularis-specific antibody can be used to detect L. intracellularis-specific polypeptides (e.g., in a cellular lysate or cell supernatant) in order to evaluate the presence or absence of the L. intracellularis-specific polypeptides. Anti-L. intracellularis-specific antibodies can be used diagnostically to detect L. intracellularis-specific polypeptides, and hence, L. intracellularis, in a biological sample, e.g., to determine the infection status of an animal, or to determine the efficacy of a given treatment regimen.

Methods of Detecting L. intracellularis

The L. intracellularis-specific nucleic acid molecules and polypeptides, and the anti-L. intracellularis-specific antibodies described herein can be used in diagnostic assays for the detection of L. intracellularis. Diagnostic assays for determining the presence or absence of L. intracellularis are performed using a biological sample (e.g., a fecal sample) to determine whether an animal has been exposed to or is infected with L. intracellularis. An exemplary method for detecting the presence or absence of L. intracellularis in a biological sample involves obtaining a biological sample from an animal and contacting the biological sample with an appropriate agent capable of detecting L. intracellularis-specific nucleic acids or polypeptides, or anti-L. intracellularis-specific antibodies.

The term “biological sample” is intended to include cells and biological fluids obtained from an animal. In one embodiment, a biological sample contains polypeptides from the animal. Alternatively, the biological sample can contain nucleic acid molecules from the animal, or the biological sample can contain antibodies from the animal. It should be understood that any biological sample in which L. intracellularis-specific nucleic acids or polypeptides, or anti-L. intracellularis-specific antibodies may be present can be utilized in the methods described herein.

In one embodiment, an agent for detecting the presence or absence of L. intracellularis in a biological sample is an isolated L. intracellularis-specific nucleic acid molecule of the invention. The presence of L. intracellularis-specific nucleic acids in a sample indicates the presence of L. intracellularis in the sample. Methods for detecting nucleic acids include, for example, PCR and nucleic acid hybridizations (e.g., Southern blot, Northern blot, or in situ hybridizations). Specifically, an agent can be one or more oligonucleotides (e.g., oligonucleotide primers) capable of amplifying L. intracellularis-specific nucleic acids using PCR. PCR methods generally include the steps of collecting a biological sample from an animal, isolating nucleic acid (e.g., DNA, RNA, or both) from the sample, and contacting the nucleic acid with one or more oligonucleotide primers that hybridize(s) with specificity to L. intracellularis-specific nucleic acid under conditions such that amplification of the L. intracellularis-specific nucleic acid occurs if L. intracellularis is present. In the presence of L. intracellularis, an amplification product corresponding to the L. intracellularis-specific nucleic acid is produced. Conditions for amplification of a nucleic acid and detection of an amplification product are known to those of skill in the art (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188). Modifications to the original PCR also have been developed. For example, anchor PCR, RACE PCR, or ligation chain reaction (LCR) are additional PCR methods known in the art (see, e.g., Landegran et al., 1988, Science, 241:1077-1080; and Nakazawa et al., 1994, Proc. Natl. Acad. Sci. USA, 91:360-364).

Alternatively, an agent for detecting L. intracellularis-specific nucleic acids can be a labeled oligonucleotide probe capable of hybridizing to L. intracellularis-specific nucleic acids on a Southern blot. An oligonucleotide probe can be, for example, a L. intracellularis-specific nucleic acid molecule such as a nucleic acid molecule having the sequence shown in SEQ ID NO:1-62 or 131-8727, or a fragment thereof. In the presence of L. intracellularis, a hybridization complex is produced between L. intracellularis nucleic acid and the oligonucleotide probe. Hybridization between nucleic acid molecules is discussed in detail in Sambrook et al. (1989, Molecular Cloning: A Laboratory Manual, 2^{nd} Ed., Cold Spring Harbor Laboratory Press, Cold Spring Harbor, N.Y.; Sections 7.37-7.57, 9.47-9.57, 11.7-11.8, and 11.45-11.57).

For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations and washes can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids, for example, from L. intracellularis and at least one organism other than L. intracellularis, under identical hybridization, washing, and exposure conditions. Preferably, the target nucleic acids (e.g., nucleic acids from L. intracellularis and at least one organism other than L. intracellularis) are on the same membrane. Representative Southern blot conditions are described in Example 9.

A nucleic acid molecule is deemed to hybridize to L. intracellularis nucleic acids but not to nucleic acids from an organism other than L. intracellularis if hybridization to nucleic acid from L. intracellularis is at least 5-fold (e.g., at least 6-fold, 7-fold, 8-fold, 9-fold, 10-fold, 20-fold, 50-fold, or 100-fold) greater than hybridization to nucleic acid from an organism other than L. intracellularis. The amount of hybridization can be quantitated directly on a membrane or from an autoradiograph using, for example, a Phosphorimager or a Densitometer (Molecular Dynamics, Sunnyvale, Calif.). It can be appreciated that useful primers and probes of the invention include primers and probes that anneal and hybridize, respectively, to nucleic acids of organisms other than L. intracellularis provided that such nucleic acids are not typically present in the relevant test animals. For example, the fact that a particular primer or probe anneals or hybridizes, respectively, to human nucleic acid does not diminish the value of that primer or probe for detecting the presence or absence of M. paratuberculosis in ruminants, since ruminants typically are not contaminated with human nucleic acid.

In addition, anti-L. intracellularis-specific antibodies provided by the invention can be used as agents to detect the presence or absence of L. intracellularis-specific polypeptides in a biological sample. The presence of L. intracellularis-specific polypeptides is an indication of the presence of L. intracellularis in the sample. Techniques for detecting L. intracellularis-specific polypeptides include enzyme linked immunosorbent assays (ELISAs), Western blots, immunoprecipitations and immunofluorescence. An antibody of the invention can be polyclonal or monoclonal, and usually is detectably labeled. An antibody having specific binding affinity for a L. intracellularis-specific polypeptide can be generated using methods described herein. The antibody can be attached to a solid support such as a microtiter plate using methods known in the art (see, for example, Leahy et al., 1992, BioTechniques, 13:738-743). In the presence of L. intracellularis, an antibody-polypeptide complex is formed.

In addition, L. intracellularis-specific polypeptides of the invention can be used as an agent to detect the presence or absence of anti-L. intracellularis-specific antibodies in a biological sample. The presence of anti-L. intracellularis-specific antibodies in a sample indicates that the animal from which the sample was obtained mounted an immune response toward L. intracellularis. Given the etiology of L. intracellularis in its host animals, an animal that has detectable levels of anti-L. intracellularis-specific antibodies is likely infected with L. intracellularis. Alternatively, an animal that is positive for anti-L. intracellularis-specific antibodies may have resisted infection following a previous exposure to L. intracellularis, or may possess maternally transmitted anti-L. intracellularis-specific antibodies. Techniques for detecting anti-L. intracellularis-specific antibodies in a biological sample include ELISAs, Western blots, immunoprecipitations, and immunofluorescence. A L. intracellularis-specific polypeptide can be attached to a solid support such as a microtiter plate by known methods (Leahy et al., supra). In the presence of L. intracellularis, a polypeptide-antibody complex is formed.

Detection of an amplification product, a hybridization complex, an antibody-polypeptide complex, or a polypeptide-antibody complex is usually accomplished by detectably labeling the respective agent. The term “labeled” with regard to an agent (e.g., an oligonucleotide, a polypeptide, or an antibody) is intended to encompass direct labeling of the agent by coupling (i.e., physically linking) a detectable substance to the agent, as well as indirect labeling of the agent by reactivity with another reagent that is directly labeled with a detectable substance. Detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Examples of indirect labeling include using a fluorescently labeled secondary antibody to detect an appropriate agent (e.g., a primary antibody), or end-labeling an agent with biotin such that it can be detected with fluorescently labeled streptavidin.

In another embodiment, the methods further involve obtaining a biological sample from an animal known to be infected with L. intracellularis (positive control) and a non-infected (negative control) animal, contacting the control samples with an agent capable of detecting L. intracellularis-specific nucleic acids or polypeptides, or anti-L. intracellularis-specific antibodies, such that the presence or absence of L. intracellularis-specific nucleic acids or polypeptides, or anti-L. intracellularis-specific antibodies in the samples is determined. The presence or absence of L. intracellularis-specific nucleic acids or polypeptides, or anti-L. intracellularis-specific antibodies in the control samples should correlate with the presence and absence of L. intracellularis in the positive and negative control samples, respectively.

Methods of Preventing a L. intracellularis Infection

In one aspect, the invention provides methods for preventing a disease or condition associated with infection by L. intracellularis (e.g., proliferative enteropathy) in an animal by administering a compound to the animal that immunizes the animal against L. intracellularis infection. Animals at risk for L. intracellularis infection can be administered the compound prior to the manifestation of symptoms that are characteristic of a L. intracellularis infection, such that a L. intracellularis infection is prevented or delayed in its progression.

In one embodiment, a compound that immunizes an animal can be a L. intracellularis-specific polypeptide. The sequences of representative L. intracellularis-specific polypeptides are disclosed herein (e.g., SEQ ID NOs:63-124) and can be produced using methods described herein. An L. intracellularis-specific polypeptide can be a fusion polypeptide, for example a L. intracellularis-specific polypeptide-immunoglobulin fusion polypeptide in which all or part of a L. intracellularis-specific polypeptide is fused to sequences derived from a member of the immunoglobulin family. An L. intracellularis-specific polypeptide or fusion polypeptide of the invention can be used as an immunogen to elicit anti-L. intracellularis-specific antibodies in an animal, thereby immunizing the animal.

In another embodiment, a compound that immunizes an animal can be a L. intracellularis-specific nucleic acid molecule. A L. intracellularis-specific nucleic acid molecule used to immunize an animal can include one of the L. intracellularis-specific nucleic acid molecules having the sequence shown in SEQ ID NOs:1-62 or 131-8727. L. intracellularis-specific nucleic acid coding sequences (e.g., full-length or otherwise) can be introduced into an appropriate expression vector such that a L. intracellularis-specific polypeptide or fusion polypeptide is produced in the animal upon appropriate expression of the expression vector. Expression of the L. intracellularis-specific nucleic acid molecule and production of a L. intracellularis-specific polypeptide in an animal thereby elicits an immune response in the animal and thereby immunizes the animal.

Compounds that can be used in immunogenic compositions of the invention (e.g., L. intracellularis-specific nucleic acid molecules or L. intracellularis-specific polypeptides) can be incorporated into pharmaceutical compositions suitable for administration. Such compositions typically comprise the nucleic acid molecule or polypeptide, and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., ingestion or inhalation), transdermal (topical), transmucosal, and rectal administration. Solutions or suspensions used for parenteral, intradermal, or subcutaneous application can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampoules, disposable syringes or multiple dose vials made of glass or plastic.

Oral compositions generally include an inert diluent or an edible carrier. Oral compositions can be liquid, or can be enclosed in gelatin capsules or compressed into tablets. Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Transmucosal administration can be accomplished through the use of nasal sprays or suppositories. For transdermal administration, the active compounds are formulated into ointments, salves, gels, or creams as generally known in the art.

It is especially advantageous to formulate oral or parenteral compositions in dosage unit form for ease of administration and uniformity of dosage. Dosage unit form as used herein refers to physically discrete units suited as unitary dosages for an animal to be treated; each unit containing a predetermined quantity of active compound calculated to produce the desired therapeutic effect in association with the required pharmaceutical carrier. The dosage unit forms of the invention are dependent upon the amount of a compound necessary to immunize the animal. The amount of a compound necessary to immunize an animal can be formulated in a single dose, or can be formulated in multiple dosage units. Immunization of an animal may require a one-time dose, or may require repeated doses.

For polypeptide vaccines, the dose typically is from about 0.1 mg/kg to about 100 mg/kg of body weight (generally, about 0.5 mg/kg to about 5 mg/kg). Modifications such as lipidation (Cruikshank et al., 1997, J. Acquired Immune Deficiency Syndromes and Human Retrovirology, 14:193) can be used to stabilize polypeptides and to enhance uptake and tissue penetration. For nucleic acid vaccines, the dose administered will depend on the level of expression of the expression vector. Preferably, the amount of vector that produces an amount of a L. intracellularis-specific polypeptide from about 0.1 mg/kg to about 100 mg/kg of body weight is administered to an animal.

Articles of Manufacture of the Invention

The invention encompasses articles of manufacture (e.g., kits) for detecting the presence of L. intracellularis-specific nucleic acids or polypeptides, or anti-L. intracellularis-specific antibodies in a biological sample (a test sample). Such kits can be used to determine if an animal has been exposed to, or is infected with, L. intracellularis. For example, a kit of the invention can include an agent capable of detecting L. intracellularis-specific nucleic acids or polypeptides, or anti-L. intracellularis-specific antibodies in a biological sample (e.g., a L. intracellularis-specific oligonucleotide, an anti-L. intracellularis-specific antibody, or a L. intracellularis-specific polypeptide, respectively).

For antibody-based kits to detect L. intracellularis-specific polypeptides, the kit can include, for example, a first antibody (e.g., attached to a solid support) that has specific binding affinity for a L. intracellularis-specific polypeptide and, optionally, a second antibody which binds to L. intracellularis-specific polypeptides or to the first antibody and is detectably labeled. For oligonucleotide-based kits to detect L. intracellularis-specific nucleic acids, the kit may comprise, for example, one or more oligonucleotides. For example, a kit of the invention can include a detectably labeled oligonucleotide probe that hybridizes to a L. intracellularis-specific nucleic acid molecule or a pair of oligonucleotide primers for amplifying a L. intracellularis-specific nucleic acid molecule. Such oligonucleotides provided in a kit of the invention can be detectably labeled or, alternatively, the components necessary for detectably labeling an oligonucleotide can be provided in the kit. Polypeptide-based kits for detecting anti-L. intracellularis-specific antibodies in a biological sample can contain a L. intracellularis-specific polypeptide as disclosed herein (e.g., attached to a solid support) and, optionally, an antibody that binds to L. intracellularis-specific polypeptides or to an anti-L. intracellularis-specific antibody and is detectably labeled.

Kits can include additional reagents (e.g., buffers, co-factors, or enzymes) as well as reagents for detecting the agent (e.g., labels or other detection molecules), as well as instructions for using such agents and reagents to detect the presence or absence of L. intracellularis-specific nucleic acids or polypeptides, or anti-L. intracellularis-specific antibodies. The kit can also contain a control sample or a series of control samples that can be assayed and compared to the biological sample. Each component of the kit is usually enclosed within an individual container and all of the various containers are within a single package.

The invention also encompasses articles of manufacture (e.g., vaccines) for preventing L. intracellularis infection in an animal. Articles of manufacture of the invention can include pharmaceutical compositions containing either a L. intracellularis-specific nucleic acid molecule or a L. intracellularis-specific polypeptide. Such nucleic acid molecules or polypeptides are formulated for administration as described herein, and are packaged appropriately for the intended route of administration. Pharmaceutical compositions of the invention further can include instructions for administration.

The invention will be further described in the following examples, which do not limit the scope of the invention described in the claims.

### EXAMPLES

### Example 1

### L. intracellularis Isolate

L. intracellularis VPB4 represents an isolate of the bacterium recovered from a pig during an outbreak of proliferative hemorrhagic enteropathy (PE) in the United States. This isolate can grow well and to relatively high titers in cell cultures in the laboratory. Vials of L. intracellularis VPB4 were maintained in sucrose-potassium glutamate (SPG; pH 7.0) solution containing 0.218 M sucrose, 0.0038 M KH₂PO₄, 0.0072 M K₂HPO₄ and 0.0049 M potassium glutamate plus 10% fetal bovine serum (FBS; Sigma, St. Louis, Mo.) at −80° C.

### Example 2

### Cultivation of L. intracellularis

Murine fibroblast-like McCoy cells (ATCC CRL 1696) were grown in Dulbecco's Modified Eagles Media (DMEM; Gibco Invitrogen Corporation, Carlsbad, Calif.) with 1% L-glutamine (Gibco Invitrogen Corporation) and 5% FBS, without antibiotics, at 37° C. in 5% CO₂. Briefly, McCoy cells were trypsinised and 5×10⁴ cells were seeded into a 175 cm² flask and incubated overnight at 37° C. in 5% CO₂. After rapidly thawing at 37° C., about 10⁴ L. intracellularis VPB4 organisms were diluted in DMEM with 1% L-glutamine and 7% FBS before being added to this 175 cm² flask containing about 30% confluent monolayer of McCoy cells. The flask was then placed in a container which was evacuated to 500 mm Hg and refilled with medical grade hydrogen and then incubated in a microaerophilic atmosphere of 8% O₂, 8.8% CO₂ and 83.2% N₂ at 37° C. The medium was replaced again 2 and 4 days after infection and the infection was harvested 7 days post inoculation for passage. The level of infection was assessed before each passage by scraping a small area of the McCoy cell monolayer from the infected flask, transferring those cells to a clean glass slide, acetone fixing them and staining by indirect immunoperoxidase using a monoclonal antibody specific for L. intracellularis (McOrist et al., 1987, Vet. Rec., 121:421-422).

The passage of infected cells was performed by treatment with 0.1% potassium chloride followed by removal of the cells from the flask with a cell scraper. Scraped cells were ruptured by passage six times through a 20-gauge needle and used to infect fresh McCoy cells in 175 cm² flasks.

### Example 3

### Purification of L. intracellularis

The monolayer of McCoy cells highly infected with L. intracellularis was harvested and the infection was passed weekly into 175 cm² flasks, using the same technique described above. Once the monolayer was 100% infected, the number of flasks containing L. intracellularis infected McCoy monolayer was tripled weekly for three weeks when bacteria present in the supernatant were combined and centrifuged for 20 minutes at 150×g to pellet any McCoy cells present in the cell culture supernatant. The bacterial cells were then centrifuged for 30 minutes at 3,400×g and the resultant L. intracellularis pellet was washed three times with PBS and stored at 4° C.

### Example 4

### Construction of a Random Small Insert Library of L. intracellularis

L. intracellularis cells were resuspended with TES buffer (50 mM Tris, 250 mM EDTA, 200 mM NaCl, pH 7.6). The suspension was mixed with an equal volume of 1.3% low melt preparative grade agarose (Bio-Rad Laboratories, Richmond, Calif.) in TES buffer and aliquoted into plug molds. Subsequent treatments with lysozyme and proteinase K were performed as previously described (Maslow et al., 1993, Diagnostic Molecular Microbiology, American Society of Microbiology, 563-72) and DNA in agarose plugs was digested with Sau3A1 (New England Biolabs, Beverly, Mass.) and separated by gel electrophoresis. The resulting fragments in the range of 0.8-2.0 kb were gel-purified with QIAEX II gel extraction kit (Qiagen, Valencia, Calif.) and then cloned into a BamH1-restricted, calf Intestinal alkaline phosphatase-treated pUC18 vector (Pharmacia, Piscataway, resulting library was >90% recombinant and contained more than 50,000 independent recombinant clones.

### Example 5

### Sequencing of L. intracellularis

As a source of template for sequencing, the small insert total genomic library described above was used. Approximately 300 recombinant clones were sequenced using M13 reverse and forward primers and ABI model 377 automated DNA sequencers (Applied Biosystems, Foster City, Calif.) at the Advanced Genetic Analysis Center (AGAC) at the University of Minnesota. Sequence data analyzing and editing were performed with public-domain software (phred, phrap, and consed; http://www.genome.washington.edu/UVGC/protocols/). Similarity searches were performed with BLASTn and BLASTx analysis using a local peptides database, which included non-redundant GenBank, SwissProt, OWL, TrEMBL, PIR, and NRL databases. The aligned nucleotide sequences were visually inspected, and the genes were assigned with known or putative functions based on similarity searches.

### Example 6

### Electron Microscopy of L. intracellularis

For examination of L. intracellularis by transmission electron microscopy, bacteria from 7-day cell culture supernatants were pelleted by centrifugation for 30 min at 3400×g and washed once with PBS. Bacteria were adsorbed onto Formvar-coated copper grids (Electron Microscopy Sciences, Fort Wash., Pa.) for 5 min and fixed on a drop of 0.5% glutaraldehyde for 2 min. The grids were then washed 3 times with distilled water for 10 sec each wash, negatively stained with 3% phosphotungstic acid (pH 6.8), and examined with a transmission electron microscope (Jeol 1200EX, Jeol USA, Inc., Peabody, Mass.).

### Example 7

### Nucleotide Sequence Accession Numbers

The nucleotide sequences of numerous L. intracellularis genes described in this study were deposited in the GenBank/EMBL nucleotide sequence data library and assigned Accession Numbers BH795457 through BH795518.

### Example 8

### Representative Sequences Identified

A total of 498 sequencing reactions were completed initially, with an average number of 632 bases per sequence reaction. This resulted in the generation of over 386,616 bp of total sequence representing 282,699 bp of unique (non-overlapping) L. intracellularis genomic DNA sequence or nearly 15% of the entire genomic sequence of this pathogen (Table 6). Comparison of the 498 L. intracellularis sequences with sequences from SwissProt or other sequences deposited in GenBank's microbial database indicates that only a small minority of sequences (n=82; 17%) had orthologs in the public sequence databases. The orthologs were from genera such as included Aquiflex, Bacillus, Escherishcia, Hemophilus, Helicobacter, Mycobacterium, Pseudomonas, Snechncystis, Treponema, Desulfovibrio, and others. A complete listing of all of the orthologs in the databases along with predicted function, Accession Number, and the species from which the closest ortholog originates are presented in Table 7.

TABLE 6
Summary of random sequencing of the small-insert total
genomic L. intacellularis library

| | |
|---|---|
| Total sequencing reactions | 498 |
| Average number of bases/sequencing reaction | 632 |
| Total number of bases obtained | 386,616 |
| Total number of unique bases of DNA | 282,699 |
| Number of matches to known proteins^{a} | 82 (17%) |

^{a}Threshold for significant homology; smallest probability <1.0e−10 using BLASTX on non-redundant GenBank, SwissProt, OWL, TrEMBL, PIR and NRL.

TABLE 7
Sequence similarities between L. intracellularis sequences and
sequences in public databases.^{a}

| | | | | |
|---|---|---|---|---|
| Predicted function | Gene | Accession No. | Species | P(n) |

I Cell envelope and cellular processes
I. 1 Cell structure

| | | | | |
|---|---|---|---|---|
| Rod shape-determining protein | rodA | SWP O83514 | Treponema pallidum | 2.00E−23 |
| Penicillin-binding protein | pbpAl | PIR C71661 | Rickettsia prowazekii | 3.00E−18 |
| Penicillin-binding protein 3 | | PIR S54872 | Pseudomonas aeruginosa | 1.00E−14 |
| Penicillin-binding protein 1A | pbpA | SWP P02918 | Escherichia coli | 1.00E−20 |
| Protective surface antigen D15 | | EMB O25369 | Helicobacter pylori | 3.00E−14 |
| Membrane protein | | PIR H70597 | Mycobacterium tuberculosis | 4.00E−13 |

I. 2 Transport/binding proteins and lipoproteins

| | | | | |
|---|---|---|---|---|
| Membrane bound Yop protein | pcrD | PIR O30536 | Pseudomonas aeruginosa | 3.00E−33 |
| GTP binding protein | lepA | SWP P74751 | Synechocystis sp. | 4.00E−41 |
| Tellurite resistance protein | tehA | SWP P25396 | Escherichia coli | 2.00E−11 |
| ABC-transporter | tycD | PIR T31077 | Brevibacillus brevis | 1.00E−13 |
| PSCJ precursor | pscJ | EMB P95438 | Pseudomonas aeruginosa | 5.00E−13 |

I. 3 Membrane bioenergetics (electron transport chain)

| | | | |
|---|---|---|---|
| Proton ATPase beta subunit | EMB Q46585 | Desulfovibrio vulgaris | 1.00E−37 |

I. 4 Mobility and chemotaxis

| | | | | |
|---|---|---|---|---|
| Flagellar hook basal body protein | flgG | PIR C70372 | Aquifex aeolicus | 3.00E−24 |
| Flagellum-specific ATP synthase | | EMB O06682 | Treponema denticola | 3.00E−28 |
| Flagellar hook-associated protein | flgK | PIR E71297 | Treponema pallidum | 3.00E−11 |

I. 5 Protein secretion

| | | | | |
|---|---|---|---|---|
| Preprotein translocase SECA subunit | secA | SWP Q55709 | Synechocystis sp. | 8.00E−27 |
| Signal recognition particle protein | | SWP P37105 | Bacillus subtilis | 9.00E−25 |

I. 6 Cell division

| | | | | |
|---|---|---|---|---|
| Cell division protein FTSA | ftsA | SWP P47203 | Pseudomonas aeruginosa | 2.00E−11 |
| Cell division protein FTSH | ftsH | SWP P71377 | Haemophilus influenzae | 4.00E−42 |
| Cell division protein ALGI | algI | EMB O52196 | Azotobacter vinelandii | 9.00E−14 |

II Intermediary metabolism
II. 1 Metabolism of carbohydrates

| | | | | |
|---|---|---|---|---|
| Thioredoxin peroxidase | ytgI | PIR F69992 | Bacillus subtilis | 1.00E−26 |
| Pyruvate-ferredoxin oxidoreductase | | EMB P94692 | Desulfovibrio vulgaris | 4.00E−34 |

II. 2 Metabolism of amino acids and related molecules

| | | | | |
|---|---|---|---|---|
| Dihydroorotase | pyrC | PIR B70959 | Mycobacterium tuberculosis | 3.00E−14 |
| Porphobilinogen deaminase | | EMB O34090 | Pseudomonas aeruginosa | 2.00E−20 |
| Uroporphyrin-III C-methyltransferase | | SWP P29928 | Bacillus megaterium | 1.00E−25 |
| D-alanine-D-alanine ligase | ddlB | SWP P44405 | Haemophilus influenzae | 6.00E−15 |
| Glutamate decarboxylase alpha | dceA | SWP P80063 | Escherichia coli | 1.00E−44 |

II. 3 Metabolism of nucleotides and nucleic acids

| | | | | |
|---|---|---|---|---|
| Exodeoxyribonuclease V, alpha | | PIR D71564 | Chlamydia trachomatis | 9.00E−16 |
| CTP synthetase | pyrG | SWP P96351 | Mycobacterium tuberculosis | 2.00E−22 |
| Glutamyl-tRNA amidotransferase subunit | | PIR B70342 | Aquifex aeolicus | 3.00E−26 |
| Endonuclease I precursor | | SWP P25736 | Escherichia coli | 1.00E−17 |
| O-sialoglycoprotein endopeptidase | | SWP O66986 | Aquifex aeolicus | 5.00E−22 |
| dTDP-glucose 4, 6-dehydratase | | PIR H69105 | Methanobacterium | 1.00E−21 |
| | | | thermoautotrophicum | |
| Thiamine-phosphate pyrophosphorylase | | SWP P72965 | Synechocystis sp. | 4.00E−16 |

II. 4 Metabolism of lipids

| | | | | |
|---|---|---|---|---|
| 3-oxoacyl-(acyl-carrier protein) reductase | fabG | SWP O67610 | Aquifex aeolicus | 1.00E−25 |

III Information pathways
III. 1 DNA synthesis

| | | | | |
|---|---|---|---|---|
| DNA topoisomerase | | SWP P06612 | Escherichia coli | 1.00E−15 |
| DNA polymerase III subunit tau and | dnaX | SWP P43746 | Haemophilus influenzae | 1.00E−15 |
| gamma | | | | |

III. 2 RNA synthesis

| | | | | |
|---|---|---|---|---|
| ATP-dependent RNA helicase | srmB | SWP P21507 | Escherichia coli | 1.00E−53 |
| DNA-directed RNA polymerase, beta | | EMB AAF07229 | Salmonella typhimurium | 2.00E−18 |
| chain | | | | |
| Transcription antitermination protein | nusG | SWP P16921 | Escherichia coli | 5.00E−16 |

III. 3 Protein synthesis

| | | | | |
|---|---|---|---|---|
| 50S ribosomal protein L1 | | PIR C70466 | Aquifex aeolicus | 2.00E−27 |
| Prolyl-tRNA synthetase | | GEN 303557 | Escherichia coli | 1.00E−12 |
| Arginyl-tRNA synthetase | argS | SWP P46906 | Bacillus subtilis | 7.00E−11 |
| Tyrosyl-tRNA synthetase | tyrS | SWP P56417 | Helicobacter pylori | 7.00E−46 |
| Cysteine-tRNA ligase | | PIR D71108 | Pyrococcus horikoshii | 2.00E−17 |
| Proline-tRNA ligase | drpA | PIR B64744 | Escherichia coli | 4.00E−19 |

IV Other functions
IV. 1 Adaptation to atypical conditions

| | | | |
|---|---|---|---|
| ATP-dependent protease LA2 | SWP P36774 | Myxococcus xanthus | 1.00E−29 |

IV. 2 Phage-related functions

| | | | |
|---|---|---|---|
| Bacteriophage Mu major head subunit | EMB AAF01112 | Bacteriophage Mu | 9.00E−14 |

IV. 3 Miscellaneous

| | | | | |
|---|---|---|---|---|
| CAPL protein | capL | SWP P94692 | Staphylococcus aureus | 1.00E−30 |
| Heat shock protein HSLV precusor | hslV | SWP P39070 | Bacillus subtilis | 7.00E−15 |
| HYPF protein | hypF | EMB O07039 | Rhizobium leguminosarum | 5.00E−17 |
| Rubredoxin-like protein | | EMB P94698 | Desulfovibrio vulgaris | 4.00E−16 |

V. Hypothetical proteins

| | | | |
|---|---|---|---|
| Hypothetical protein MJ1665 | PIR G64507 | Methanococcus jannaschii | 2.00E−17 |
| Hypothetical 29.2 KD protein | SWP P37545 | Bacillus subtilis | 4.00E−18 |
| Hypothetical 15.6 KD protein | SWP Q99342 | Escherichia coli | 4.00E−11 |
| Hypothetical 70.4 KD protein | SWP P54123 | Synechocystis sp. | 2.00E−11 |
| Hypothetical 34.6 KD protein | SWP P45476 | Escherichia coli | 2.00E−17 |
| Hypothetical 45.9 KD protein | SWP P71607 | Mycobacterium tuberculosis | 3.00E−18 |
| Hypothetical protein sir 1117 | PIR S74480 | Synechocystis sp. | 1.00E−11 |
| Hypothetical 37.1 KD protein | EMB O69560 | Streptomyces coelicolor | 5.00E−17 |
| Hypothetical 34.6 KD protein | GEN P45476 | Escherichia coli | 4.00E−14 |
| Hypothetical protein 2 | PIR S60064 | Corynebacterium glutamicum | 1.00E−11 |

^{a}Threshold for significant homology; smallest sum probability <1.0e⁻¹⁰ using BLASTX on non-redundant GenBank, SwissProt, OWL, TrEMBL, PIR, and NRL.

As can be noted from Table 7, L. intracellularis contains sequences that exhibit homology to sequences from all three domains of life; Archaea, Bacteria, and Eukarya. The most common orthologs were from Bacteria, including Gram-negative as well as Gram-positive organisms and bacteria with a widely disparate level of G+C content.

The random sequencing approach identified several sequences in L. intracellularis that are of interest from a diagnostic, genetic, virulence or immunoprophylaxis standpoint. For instance, genes homologous to those encoding proteins involved in flagellar biosynthesis and assembly have been identified in our preliminary screen of the L. intracellularis genome (Table 7). These findings provide confirmation of the recent observations that some isolates of L. intracellularis possess a single polar flagellum. These observations are consistent with the fact that L. intracellularis displays a darting or directed motion when visualized in active cultures, and suggest a molecular mechanism by which the bacterium may accomplish this activity. Importantly, the identification of a flagellum in isolates of L. intracellularis, coupled with the sequences that correspond to regions of genes involved in flagellar assembly, provides us with a facile means of developing specific reagents to delineate its role in virulence and infectivity. It is noteworthy that flagellar structures are often highly immunoreactive, and it is well recognized from a variety of model systems that antibodies against flagella structures can lead to bacterial opsonization and killing; hence these genes may also be of interest from an immunoprophylaxis standpoint.

Preliminary sequence analysis also identified a L. intracellularis homolog to a membrane-bound Yop (Yersinia outer protein). The capacity of Yersiniae (Y. pestis, Y. pseudotuberculosis, and Y. enterocolitica) to resist the immune system of their host depends on the Yop virulon. This system allows extracellular bacteria adhering to the surface of eukaryotic cells to inject bacterial proteins into the cytosol of target cells in order to disarm them or disrupt their communications. Some Yops (e.g., effector Yops) may be injected directly into the target cells through a system known as type III targeting. Others may be excreted into the extracellular environment or remain associated with the bacterial membranes. An example of the latter is LcrV, a 41 kDa secreted protein that was described in the mid-1950s as a protective antigen of the plague bacillus, Y. pestis. LcrV is one of the major Yops that is known to be essential for virulence. While its exact role in the virulon is unclear, it is required for translocation of the others across the target cell membrane. It then helps to form a pore-like structure in the target cell membrane. Homologs of LcrV have been found in numerous bacterial pathogens that use this type III secretion mechanism for invasion or pathogenicity, including Salmonella sp. and Pseudomonas aeruginosa. Interestingly, the preliminary sequence analysis of L. intracellularis identified a homolog of LcrV (ortholog of the P. aeruginosa protein, PcrD), strongly suggesting that L. intracellularis is likely to contain a type III secretion system.

A third gene of potential importance in vaccine and immunodiagnostic reagent development is the L. intracellularis homolog of the major membrane protein D15 in H. pylori (also termed Oma87). The function of the D15/Oma87 protein family is not clear. D15/Oma87 has been shown, however, to have homologs and represent a major protective antigen in isolates of H. influenzae, P. multocida, and Shigella flexneri. Conservation of a homologous gene in such diverse species suggests that this gene is important. Anti-D15 antibodies were detected in eight of nine sera from patients recovering from H. influenza infection. Therefore, D15 and other newly identified targets may be of potential interest from a vaccine, diagnostic test, or drug development standpoint.

### Example 9

### DNA Hybridizations

Genomic DNA is extracted from several isolates of L. intracellularis using methods known in the art (see, for example, Diagnostic Molecular Microbiology: Principles and Applications, Persing et al. (eds), 1993, American Society for Microbiology, Wash. D.C). Briefly, Lawsonia are harvested by centrifugation at 8,000 rpm for 15 min and the pellet is resuspended in 11 ml of Qiagen buffer B1 containing 1 mg/ml Qiagen RNase A. Lipase is added (450,000 Units, Sigma Catalog No L4384) to digest cell wall lipids. Following incubation for 2 h at 37° C., 20 mg of lysozyme is added and incubation proceeds for an additional 3 h at 37° C. 500 μl of Qiagen proteinase K (20 mg/ml) is added and incubated for 1.5 h at 37° C. Qiagen buffer B2 (4 ml) is added and the slurry is mixed and incubated 16 h at 50° C. The remaining cellular debris is removed by centrifugation at 10,000 rpm for 20 min. The supernatant is poured over a pre-equilibrated Qiagen 500/G genomic tip. The loaded column is washed and processed according to the instructions of the manufacturer.

PstI restricted DNA fragments are separated on a 1% agarose gel. DNA-containing gels are depurinated, denatured, and neutralized as described by Sambrook et al. (1989, Molecular Cloning: A Laboratory Manual, Second Ed., Cold Spring Harbor Laboratory Press, Cold Spring Harbor, N.Y.). DNA is transferred by capillary action to BrightStar-Plus membranes (Ambion, Austin, Tex.) and probes are labeled using [α-³²P]dCTP (ICN, Cost Mesa, Calif.) by random priming. Hybridization is performed in a AUTOBLOT hybridization oven (Bellco Biotechnology, Vineland, N.J.) at 45° C. for 16 h in ExpressHyb hybridization solution (Clontech, Palo Alto, Calif.). Probed blots are washed sequentially with solutions increasing in stringency as follows: 2 washes at room temp in 2×SSC, 0.1% SDS; 2 washes at room temp in 0.2×SSC, 0.1% SDS; and 2 washes at room temp in 0.16×SSC, 0.1% SDS. Detection is by autoradiography at room temp using BioMax MR film (Kodak, Rochester, N.Y.) with a Kodak intensifying screen for less than 16 hours.

### Example 10

### PCR Amplification

The L. intracellularis genome was analyzed for the presence of variable number tandem repeat (VNTR) sequences using Tandem Repeat Finder software. From this analysis, four putative VNTR regions were found. Specific primer sequences were designed upstream and downstream of these regions using Primer 3 software. The sequence and positions of the primers used in the amplification reactions are shown in Table 8.

TABLE 8

| | Position | Repeat | | |
|---|---|---|---|---|
| Plasmid | Amplified | (copy #) | Left Primer | Right Primer |
| 1 | 18,951-18,988 | ATA | 5′-TTCTCA CAT | 5′-CCC CAC CTT |
| | of | (12) | TTT CAA ATC TTT | TT-3′ |
| | SEQ ID NO:8736 | | TTC-3′ | SEQ ID NO:8729 |
| | | | SEQ ID NO:8728 | |
| 3 | 194,585-194,619 | CA | 5′-TTG ACG TTA | 5′-TTG TAT ATT |
| | of | (17) | TCT TTA GCC TAC | CAA AAA GGT TCA |
| | SEQ ID NO:8738 | | CA-3′ | ATG TAA-3′ |
| | | | SEQ ID NO:8730 | SEQ ID NO:8731 |
| 3 | 130,523-130,562 | ATA | 5′-CAA CAC AAA | 5′-TCA TGC ATC |
| | of | (13) | ATA TCC CCT | GCA TCT TTT AAT |
| | SEQ ID NO:8738 | | TGG-3′ | TT-3′ |
| | | | SEQ ID NO:8732 | SEQ ID NO:8733 |
| 3 | 164,830-164,863 | CA | 5′-GGT TAC TAT | 5′-TGT GCC TGT |
| | of | (19) | TCT TAG GTT ATT | CTT TCT TGT AGT |
| | SEQ ID NO:8738 | | GCC AGA-3′ | GA-3′ |
| | | | SEQ ID NO:8734 | SEQ ID NO:8735 |

PCR amplification of L. intracellularis-specific nucleic acid molecules was performed as follows. A PCR reaction mix was generated that contained 2.5 μl of 10× buffer, 2.0 μl of a 10 mM dNTP mix, 1.0 μl of 25 mM MgCl₂, 1.0 μl L. intracellularis DNA, 3.0 μl of a 5 μM stock of the left primer, 3.0 μl of a 5 μM stock of the right primer, 0.15 μl polymerase, and 12.85 μl H₂O. The PCR reaction conditions were as follows: a 5 min denaturing step at 94° C., followed by 30 cycles of: 94° C. for 30 sec, 57° C. for 30 sec, and 72° C. for 1 min. At the end of 30 cycles, the samples were incubated at 72° C. for 10 min and then the reaction was held at 4° C. PCR amplifications generally used Taq DNA polymerase and the corresponding buffer (Roche Molecular Biochemicals, Indianapolis, Ind.).

### Example 11

### Expression of L. intracellularis Genes in E. coli

To confirm coding predictions of novel L. intracellularis genes and assess their immunogenicity, coding sequences are amplified from the genome by PCR and cloned into the pMAL-c2 E. coli expression plasmid. These proteins are expressed as a fusion with E. coli maltose binding protein (MBP) to enable affinity purification on an amylase resin column. An immunoblot is probed with a monoclonal antibody that binds MBP, which identifies each fusion protein. A duplicate immunoblot is probed with polyclonal sera from a rabbit immunized with a heat-killed preparation of L. intracellularis. Only the fusion protein containing a L. intracellularis-specific polypeptide should be detected by the rabbit sera, which indicates that the polypeptide is produced by L. intracellularis. The MBP protein was not detected by the polyclonal sera.

### Example 12

### Making a Vaccine

Coding sequences within L. intracellularis-specific DNA fragments are cloned into E. coli expression vectors (e.g., containing a sequence encoding a 6×His tag). Heterologously expressed L. intracellularis proteins are affinity purified from E. coli lysates by a polyhistidine tag. These purified proteins are then evaluated serologically with a panel of sera from infected and control pigs to determine if the protein is recognized by sera from infected animals.

Specifically, an open reading frame identified as unique to L. intracellularis is amplified from genomic DNA, cloned into the pCRT7 expression vector (Invitrogen), and transformed into E. coli DH5-α. Each of the constructs are verified by DNA sequence analysis. The level of expression of the gene of interest is evaluated by loading the recombinant E. coli lysates onto SDS-PAGE gels and staining them in Coomassie blue. Expressed proteins are purified from E. coli lysates using the vector-encoded polyhistidine tag that has affinity for metal ions. Column purification using TALON metal resin (Clontech) is used. The fusion alone is used as a negative control. Comparisons of the reactivity of a collection of pig antisera with the fusion proteins are conducted using a slot-blotting device (BioRad). Lysates of recombinant E. coli are loaded onto preparative 12% (w/v) polyacrylamide gels and transferred to nitrocellulose. After blocking, these filters are placed into the slot-blot device. Individual pig antisera, each diluted 1:200, is added to independent slots. The rest of the procedure is carried out using standard immunoblot protocols. Protein G-peroxidase diluted 1:25,000 or anti-pig IgG-peroxidase diluted 1:20,000 are used for detection of bound antibody.

### Example 13

### Production of Monoclonal and Oolyclonal Antibodies Against L. intracellularis-Specific Polypeptides

All expressed and purified L. intracellularis-specific polypeptides are used to immunize both BALB/c mice and New Zealand white rabbits. Standard immunization regimens are used in each instance. TiterMax or Freund's incomplete serve as the adjuvant. Splenic lymphocytes from the immunized mice are hybridized with myeloma cells for the production of monoclonal antibodies. ELISA is the method used to assay secreting hybridomas for reactivity to purified antigens. Hybridomas in positive wells are cloned and expanded using standard methods. Rabbit antisera is collected following boost injections of isolated polypeptide until a sufficient titer is obtained.

### Example 14

### ELISA Assays

Improvement in the specificity of the ELISA test for detection of proliferative enteropathy in animals (e.g., pigs) has always been a major goal. The purified L. intracellularis-specific polypeptide to be evaluated is diluted in PBS and added to 96-well microtiter plates. Plates with bound polypeptide are blocked in PBS containing 1% gelatin and then washed three times with PBS containing 0.05% Tween. Pig sera to be tested is diluted 1:400 in PBS, added to individual wells, and processed as a standard ELISA. Mouse anti-bovine IgM or mouse anti-bovine IgG is the second antibody in these assays. Results generally show that the use of a biotinylated second antibody followed by streptavidin/alkaline phosphatase and enzyme detection can enhance test sensitivity 8 to 16-fold (based on antibody titers) as compared to the standard direct ELISA.

For all evaluations, it is necessary to include samples from known negative animals to assess specificity. In addition, because of potential cross-reactivity that may be encountered with other bacteria, especially other L. intracellularis, sera from animals known to be naturally or experimentally infected with other L. intracellularis, are included.

### Example 15

### Use of Antibodies Against L. intracellularis-Specific Polypeptides in Immunohistochemical Diagnosis of Infected Pig Tissues

Histopathologic analysis of tissues from infected animals can be used to detect L. intracellularis. However, these methods are non-specific and do not distinguish among isolates. Therefore, pig tissues from L. intracellularis-infected and -uninfected animals are tested by histopathologic analysis using high-titer antibodies directed at L. intracellularis-specific polypeptides. Briefly, tissue samples from pigs are fixed in buffered formalin, processed routinely, and embedded in paraffin wax. 6 μm cut sections are stained with hematoxylin and eosin or Ziehl-Neelsen by conventional methods. Replicate unstained sections are prepared for immunohistochemistry. Sections that are immunostained are deparaffinized, rehydrated and blocked using routine methods (Stabel et al., 1996, J. Vet. Diagn. Invest., 8:469-73). Blocked sections are incubated with L. intracellularis-specific antibodies developed in the above-described studies. Depending on the nature of the primary antibody, either goat anti-rabbit biotinylated antibody or goat anti-mouse biotinylated antibody is added followed by washing instreptavidin-alkaline phosphatase solution. The tissue is stained with chromogen, and Histomark Red. Results are visualized under a bright-field microscope. Staining intensities are quantitatively compared among the different infected and uninfected tissues.

### Example 16

### Detection of L. intracellularis by PCR Amplification

Detection and identification of L. intracellularis isolates using oligonucleotide primers complementary to L. intracellularis-specific nucleic acid sequences was examined by PCR.

L. intracellularis isolates of geographic and temporal diversity (PHE/MN1-00, VPB4, 15540D, 963/93, foal/96, and hamster-1) were used to determine if there was inter-strain variability among isolates of L. intracellularis by amplifying VNTR regions of the genome. To assess if the VNTR profiles were conserved and stable in a specific isolate, an isolate was tested prior to cultivating in cell culture, after low- and high-passage cell culture, and after serial passage through a pig. In addition, 100 fecal samples from 4 different proliferative enteropathy outbreaks were tested by extracting genomic DNA from the fecal sample in the absence of prior cultivation. Each DNA sample was subjected to four different rounds of polymerase chain reaction (PCR) amplification using the four respective primer sets. PCR products were then sequenced using an ABI 3100 automated fluorescent DNA sequencer. The number of tandem repeats for each loci were calculated, creating a VNTR profile for each sample.

Table 9 shows that the six L. intracellularis isolates contained different numbers of each of the VNTRs examined. These results indicate, therefore, that there is identifiable genomic differences between L. intracellularis isolates. The VNTR profile of L. intracellularis obtained directly from a diseased intestine was identical to that obtained after purification and inoculation into cell culture, after low passage, and after serial passage through a pig. Thus, the VNTR regions described herein remain stable and conserved under various conditions. Samples from separate herd experiencing proliferative enteropathy outbreaks showed unique VNTR profiles; however, samples within the same outbreak shared identical profiles.

TABLE 9

| | No. of VNTRs/L. intracellularis isolate | | | | | |
|---|---|---|---|---|---|---|
| VNTR | | | | | Foal/ | |
| (Genetic Element) | 963/93 | 15540D | PHE | VPB4 | 96 | Hamster-1 |
| CA(17) | 16 | 10 | 17 | 15 | 13 | 13 |
| (3) | | | | | | |
| ATA(13) | 12 | 10 | 13 | 11 | 5 | 5 |
| (3) | | | | | | |
| CA(19) | 16 | 17 | 19 | 16 | 16 | 13 |
| (3) | | | | | | |
| ATA(12) | 9 | 8 | 12 | 9 | 10 | 13 |
| (1) | | | | | | |

VNTRs contain a high level of polymorphism, resulting in a high discriminatory capacity. Based on results in the present study, analysis of VNTR profiles appears to be a useful tool for distinguishing between strains or isolates of L. intracellularis. The assay proved to be robust and gave identical results upon repeat analysis. This method of rapidly detecting L. intracellularis and tracing specific isolates may be used epidemiologically to allow rapid identification of the source of an infection and thereby reduce the rate of transmission.

### Example 17

### Annotation of L. intracellularis Genetic Elements

The sequencing and assembly strategies used herein for L. intracellularis were as described for Pasteurella multocida (see May et al., 2001, Proc. Natl. Acad. Sci. USA, 98:3460-5). For these studies, assembled L. intracellularis contig fragments greater than 10 kb were chosen. Predicted coding sequences were identified using ARTEMIS software and TB-parse (Cole et al., 1998, Nature, 393:537-44). The TB-parse results were compared and verified manually in ARTEMIS. A putative ribosome-binding site (RBS) was also evaluated for each coding sequence. The presence of an AG-rich sequence approximately 30-bp upstream of the start codon was scored as a putative RBS sequence. Similarities were identified with BLASTP analysis by using GenBank and a local database constructed by the Computational Biology Center at the University of Minnesota (http://www.cbc.umn.edu).

ARTEMIS and ACT are funded by the Wellcome Trust's Beowulf Genomics initiative and are available free on the internet at http://www.sanger.ac.uk/Software/. Sequence alignments to produce figures or schematic illustrations were performed with AssemblyLIGN™ software (Accelrys, Princeton, N.J.).

### Example 18

### Analysis of the L. intracellularis Genome

A shotgun strategy was adopted to sequence the genome of L. intracellularis. To create a library having an insert size of 1.5- to 3.0-kb, genomic DNA from a L. intracellularis PHE isolate was isolated using a chloroform/cetyltrimethylammonium bromide-based method and DNA was sheared by nebulization and cloned into a pUC18 plasmid vector for shotgun sequence analyses essentially as described (May et al., 2001, Proc. Natl. Acad. Sci., USA, 98:3460-5). The resulting clones were sequenced from both ends using Dye-terminator chemistry on ABI 3700 and 3100 (Applied Biosystems) sequencing machines. Sequence assembly and verification were accomplished by using the phredPhrap and Consed suite of software (http://genome.washington.edu). In order to close the final gaps at the end of the shotgun phase, several methods were used, including primer walking and random PCR. The final sequence showed that the L. intracellularis genome consisted of 4 genetic elements (3 plasmids and 1 chromosome).

The sequence of each L. intracellularis genetic element is shown in Tables 10, 11, 12, and 13, which are contained on the appended compact disc, which has been incorporated by reference herein. Table 10 contains the sequence of plasmid 1 (genetic element 1; SEQ ID NO:8736), which is 27,048 nt in length and has a % GC content of 29.05%. Table 11 contains the sequence of plasmid 2 (genetic element 2; SEQ ID NO:8737), which is 39,794 nt in length and has a % GC content of 29.23%. Table 12 contains the sequence of plasmid 3 (genetic element 3; SEQ ID NO:8738), which is 194,553 nt in length and has a % GC content of 32.91%. Table 13 contains the sequence of the chromosome (genetic element 4; SEQ ID NO:8739), which is 1,457,619 nt in length and has a % GC content of 33.28%.

Potential coding sequences (CDSs) in the genome were predicted by using GLIMMER, and ARTEMIS, and the results were compared and verified manually in ARTEMIS. Tables 14, 15, 16, and 17 (contained on the appended compact disc, which has been incorporated by reference herein) describe the annotation of the L. intracellularis sequences for genetic elements 1, 2, 3, and 4, respectively. Tables 18, 19, 20, and 21 (contained on the appended compact disc, which has been incorporated by reference herein) describe the nucleotide sequence of each predicted CDS for genetic elements 1, 2, 3, and 4, respectively. Tables 22, 23, 24, and 25 (contained on the appended compact disc, which has been incorporated by reference herein) describe the predicted amino acid sequences encoded by each predicted CDS for genetic element 1, 2, 3, and 4, respectively.

### Example 19

### Real-Time PCR

A PCR master mix is prepared containing the following: 1×TaqMan Buffer A (Perkin Elmer), 5.0 mM MgCl₂, 1.25 units per reaction Amplitaq Gold, 200 μM dATP, 200 μM dCTP, 200 μM dGTP, 400 μM dUTP, 5% DMSO, 0.01 units per reaction UNG, 100 μM of each primer, and 150 μM of each probe. Five μl of template DNA is placed in each PCR reaction tube, and 45 μl of Master mix is added. PCR samples are subject to initial denaturation at 50° C. for 10 minutes and then at 95° C. for 10 minutes; 40 amplification cycles of 94° C. for 30 seconds, 60° C. for 30 seconds, and 72° C. for 1 minute; a final extension at 72° C. for 7 minutes; and a soak at 25° C. Specific PCR products are detected using the ABI Prism 7700 or 7900HT Sequence Detection System (Applied Biosystems, Inc.). Results are recorded as Delta-RQ, which is the difference in the Rn values from the samples and the no-template control. The Rn values are the ratio of reporter emission to quencher emission. Agarose gel electrophoresis with ethidium bromide staining is performed to verify the results of the TaqMan assay. All assays are performed in duplicate.

To evaluate the sensitivity of the assay, ten-fold dilutions of L. intracellularis strain PHE cells were spiked into a negative fecal sample collected from a known L. intracellularis-free pig farm. L intracellularis DNA amounts used for template range from 100 ng to 1 fg. DNA is extracted from the spiked samples using a QIAamp DNA Stool Mini Kit, and the sensitivity of the assay for detecting L intracellularis in fecal samples is assessed by PCR as described above.

The specificity of the assay is evaluated using template DNA from other Lawsonia and non-Lawsonia spp. In addition, the TaqMan assay is compared to conventional PCR.

### Example 20

### Use of Real-Time PCR for Detection and Quantitation of L. intracellularis

A real-time PCR assay is developed for detection and quantitation of L. intracellularis. Primers and probes are designed based on a novel unique sequence. To increase sensitivity, two sets of primer-probe combinations are tested and used in the TaqMan assay as a multiplex strategy to amplify fragments of the unique L. intracellularis sequence. Assay conditions are optimized for MgCl₂, primer, and probe concentrations in the reaction mix; in related experiments, optimal concentrations are found to be 5.0 mM MgCl₂, 100 nM each primer, and 150 nM each probe.

To quantitate standard L. intracellularis, curves resulting from amplification of known amounts of L. intracellularis DNA (100 ng to 1 fg) are generated. A regression line is generated from the data points, and the correlation coefficient (R²) value is determined. The ability to employ the TaqMan approach for quantitation of L. intracellularis also is determined. For example, a sample containing a “blinded” number of L. intracellularis cells can be analyzed using real-time PCR and calculations can be performed to approximate the number of cell equivalents that were spiked into the sample.

Known amounts of L. intracellularis PHE genomic DNA are used to test the sensitivity of the real-time PCR assay. DNA concentrations ranging from 100 ng to 1 fg result in Ct values. The cut-off point for accurate detection of L. intracellularis PHE DNA is determined and correlated with cell equivalents of L. intracellularis. Ten-fold dilutions of L. intracellularis PHE cells spiked in feces also are used to determine the sensitivity of the assay.

The specificity of the TaqMan assay is tested using different L. intracellularis isolates, for example, from different animal species, and isolates representing non-L. intracellularis species.

### Example 21

### Representative Nucleic Acid and Polypeptide Sequence

The following polypeptide sequence (SEQ ID NO:8740) has homology with hemolysins from Synechosystis sp. and Nostoc sp., and is encoded by the following nucleic acid sequence (SEQ ID NO:8741). The following nucleic acid sequence (SEQ ID NO:8741) contains the coding sequence as well as approximately 50 nucleotides upstream and downstream of the coding sequence.

(SEQ ID NO:8740)

MIILLGTVFLIVLISALCSMMEAAIYSIPITYIEHLREQGSKKGEKLYYLHSNDQPITA
VLILNTIANTAGAALAGAIATTTLHESTMPFFAAILTLLILAFGEIIPKTLGVAYSKRIAI
ILLNPLCILIVTLKPLIMLSSYLTRLVSPRKRPTVTEDDIRALTSLSRESGRIKPYEEHVI
KNILSLDLKYAHEIIMTPRTMVFSLHENLTVSEAYSNPKIWNYSRTPTYGENNEDITGII
QRYEIGRYMTNGETEKKLLEIIMQPAKFVLESQTVDHLLLAFLEERQHLFIVLDEYGG
LSGVVSLEDVLETMLGREIVDESDTTPDLRALAKiKRHSALIQNM(NTLLK

(SEQ ID NO:8741)

caagctataataacttacgctatgttagcagcacttctaattagagcaatttattaggacaataatcatgataatccttttaggaactgtttttc
ttattgttcttatctctgcattatgctcaatgatggaagctgctatatactctatccctattacttatattgaacaccttcgtgaacagggaagc
aaaaaaggagaaaaactttattatttacatagtaatattgatcagcctattacagccgtattaatattgaatactatagcaaatactgctgga
gctgcccttgctggagcaattgctacaacaacacttcatgaatctactatgcctttctttgcagcaatcctcaccttgcttattttagcttttgg
ggaaattatacctaaaacactaggtgttgcttactctaaacgtattgctataattctccttaatcctctctgtattcttatagttactttaaaaccc
cttattatgctttcaagctacttaacacgacttgtttcacctcgaaaacgtcctacagttacagaagatgacatccgtgcacttacaagtcttt
ccagagagtctggtcgtattaagccatatgaagaacatgtcataaaaaatatccttagtcttgatttaaaatatgctcatgaaattatgactc
ccagaactatggtcttttcacttcatgaaaaccttactgtctctgaagcttatagcaaccccaaaatatggaactatagtcgcatccctactt
atggagaaaataacgaagacattactggcattatccaacgatatgaaattggacgatatatgaccaatggagaaacagaaaaaaaactt
ttagaaattatgcaaccagcaaaatttgtccttgaaagtcaaactgtagatcatttacttcttgcatttttagaagaaagacaacatctttttatt
gtacttgatgagtatgggggattatctggtgttgtttccttagaagatgtattagaaactatgcttggaagagaaattgttgatgaaagtgat
acaacacctgatcttagagcacttgcaaaaaaaagacatagtgcattaatccaaaataataaaaatactcttttaaaataacagaaatata
cctttactctctaataagtattaatataacttaaagtgtaagctgaaacacctttcaaaataaag

The following polypeptide sequence (SEQ ID NO:8742) has homology with a hemolysin from Desulfovibrio desulfuricans, and is encoded by the following nucleic acid sequence (SEQ ID NO:8743). The following nucleic acid sequence (SEQ ID NO:8743) contains the coding sequence as well as approximately 50 nucleotides upstream and downstream of the coding sequence.

(SEQ ID NO:8742)

MAKHKVRADELVFLQGLAESREQAKRLIMAGKVTLTNNSTTIPLRLEKPGHKYPLES
ICSLIGVERFVSRGAYKILTALDFFKIDVKSCICLDAGASTGGFTDCLLQHGASKVYA
IDVGKGQLHEKLYTNEQVINIEGVNLRTASKDLWEEVDILTTDVSFISLTLILPSCIRWL
KASGIHALTKPQFELYPDKIKKGVVKETSLQYEAVEKIIHFCQSELGLIFIGVVPSVIKG
PKGNQEYLIYLKKR

(SEQ ID NO:8743)

tatgactagcaagctaatatttatgtgttatattatcactatatatttttataaataataagatgagaagaaagaatggccaaacataaagtac
gtgctgatgaacttgtttttttacaagggttagcagaaagtcgtgaacaagctaaacgacttattatggcaggtaaggttacattaactaat
aattctacaactataccattacgtttggaaaaaccaggacataaatatccattagaaagtatctgcagtttaataggggtagaacgttttgt
gagtagaggagcatataagctattaactgctctagatttttttaaaattgatgtaaaaagttgtatttgtcttgatgcaggcgcatctactggt
gggtttacagattgtcttttacaacatggagcatctaaagtatatgcgattgatgtaggcaaaggtcaattacatgagaaactgtatactaa
tgaacaagttataaatattgagggagtgaatttacgtacagcatctaaagatcttattcctgaagaagtagatattttaactattgatgtttctt
ttatatcgcttactttgattttaccgtcatgtatacgttggctaaaggcttccggaattattattgccttaataaagcctcaatttgaattatatcc
agataaaataaaaaaaggtgtagtaaaagaaactagcttgcaatatgaagcagtagaaaaaattattcatttttgtcaatcagaacttgga
cttatatttattggtgttgttccgtcggtaataaaaggtccaaaaggaaatcaagaatatcttatttacttgaaaaaacgttaataatacttatt
ataatttgtattctatattatgtaggtatataaatataaagaggtatgatta

Table 26 contains relevant information regarding SEQ ID NOs:8741 and 8743, and corresponds in content to Tables 2, 3, 4, and 5.

| SEQ | | |
|---|---|---|
| ID | N | |
| NO: | (nt) | Organisms |
| 8741 | 29 | Homo sapiens, Rattus norvegicus, Mus musculus, Clostridium tetani E88, |
| | | Clostridium sticklandii, Fusobacterium nucleatum subsp. nucleatum ATCC25586, |
| | | Streptococcus pneumoniae, Oryza sativa, Haenianthus salicifolius var. |
| | | obovatus, Haenianthus incrassatus, Danio rerio, Arabidopsis thaliana, |
| | | Drosophila melanogaster, Caenorhabditis elegans, Grapevine leafroll- |
| | | associated virus, M. capricolum, Utricularia laciniata |
| 8743 | 33 | Mus musculus, Clostridium acetobutylicum ATCC824, Plasmodium |
| | | falciparum, Homo sapiens, Cryptosporidium parvum, Danio rerio, |
| | | Melanoplus sanguinipes entomopoxvirus, Dictyostelium discoideum, |
| | | Arabidopsis thaliana, Marchantia polymorpha, E. histolytica, Ciona |
| | | intestinalis, Oryza sativa, Lotus corniculatus var. japonicus, Yaba monkey |
| | | tumor virus, Entamoeba histolytica, Drosophila melanogaster, Xenopus |
| | | laevis, Caenorhabditis elegans, Photorhabdus luminescens subsp. laumondii |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid, wherein said nucleic acid comprises a nucleic acid molecule of at least 10 nucleotides in length, said molecule having at least 75% sequence identity to SEQ ID NO:8741 or to the complement of SEQ ID NO:8741, wherein any said molecule that is 10 to 29 nucleotides in length, under standard amplification conditions, generates an amplification product from L. intracellularis nucleic acid using an appropriate second nucleic acid molecule, but does not generate an amplification product from nucleic acid of any of the organisms selected from the group consisting of Homo sapiens, Rattus norvegicus, Mus musculus, Clostridium tetani E88, Clostridium sticklandii, Fusobacterium nucleatum subsp. nucleatum ATCC 25586, Streptococcus pneumoniae, Oryza sativa, Haenianthus salicifolius var. obovatus, Haenianthus incrassatus, Danio rerio, Arabidopsis thaliana, Drosophila melanogaster, Caenorhabditis elegans, Grapevine leafroll-associated virus, M. capricolum, and Utricularia laciniata using an appropriate third nucleic acid molecule.

2. The nucleic acid of claim 1, wherein said nucleic acid molecule has the sequence shown in SEQ ID NO:8741.

3. The nucleic acid of claim 1, wherein said nucleic acid molecule has at least 75% sequence identity to SEQ ID NO:8741.

4. The nucleic acid of claim 1, wherein said nucleic acid molecule has at least 80% sequence identity to SEQ ID NO:8741.

5. The nucleic acid of claim 1, wherein said nucleic acid molecule has at least 85% sequence identity to SEQ ID NO:8741.

6. The nucleic acid of claim 1, wherein said nucleic acid molecule has at least 90% sequence identity to SEQ ID NO:8741.

7. The nucleic acid of claim 1, wherein said nucleic acid molecule has at least 95% sequence identity to SEQ ID NO:8741.

8. The nucleic acid of claim 1, wherein said nucleic acid molecule has at least 99% sequence identity to SEQ ID NO:8741.

9. A vector comprising the nucleic acid of claim 1.

10. A host cell comprising the vector of claim 9.

11. An isolated polypeptide encoded by the nucleic acid of claim 1.

12. The isolated polypeptide of claim 11, wherein said polypeptide has the amino acid sequence shown in SEQ ID NO:8740.

13. An article of manufacture, wherein said article of manufacture comprises the polypeptide of claim 11.

14. An antibody, wherein said antibody has specific binding affinity for the polypeptide of claim 11.

15. A method for detecting the presence or absence of L. intracellularis in a biological sample, comprising the steps of:
contacting said biological sample with at least one nucleic acid under standard amplification conditions, wherein said nucleic acid comprises a nucleic acid molecule of at least 10 nucleotides in length, said molecule having at least 75% sequence identity to SEQ ID NO:8741, wherein an amplification product is produced if L. intracellularis nucleic acid is present in said biological sample; and
detecting the presence or absence of said amplification product,
wherein the presence of said amplification product indicates the presence of L. intracellularis in the biological sample, and wherein the absence of said amplification product indicates the absence of L. intracellularis in the biological sample.

16. The method of claim 15, wherein said biological sample is derived from pigs, hamsters, foals, dogs, deer, fox, rabbits, rats, emus, ostriches, non-human primates, and humans.

17. The method of claim 15, wherein said biological sample is a fecal sample and a blood sample.

18. A method for detecting the presence or absence of L. intracellularis in a biological sample, comprising the steps of:
contacting said biological sample with at least one nucleic acid under hybridization conditions, wherein said nucleic acid comprises a nucleic acid molecule of at least 10 nucleotides in length, said molecule having at least 75% sequence identity to SEQ ID NO:8741, wherein a hybridization complex is produced if L. intracellularis nucleic acid is present in said biological sample; and
detecting the presence or absence of said hybridization complex,
wherein the presence of said hybridization complex indicates the presence of L. intracellularis in said biological sample, and wherein the absence of said hybridization complex indicates the absence of L. intracellularis in said biological sample.

19. The method of claim 18, wherein nucleic acids present in said biological sample are electrophoretically separated.

20. The method of claim 19, wherein said electrophoretically separated nucleic acids are attached to a solid support.

21. The method of claim 20, wherein said solid support is a nylon membrane or a nitrocellulose membrane.

22. The method of claim 18, wherein said one or more nucleic acids are labeled.

23. The method of claim 18, wherein said biological sample is selected from the group consisting of a fecal sample and a blood sample.

24. A method for detecting the presence or absence of L. intracellularis in a biological sample, comprising the steps of:
contacting said biological sample with the polypeptide of claim 11, wherein a polypeptide-antibody complex is produced if an antibody having specific binding affinity for said polypeptide is present in said sample; and
detecting the presence or absence of said polypeptide-antibody complex,
wherein the presence of said polypeptide-antibody complex indicates the presence of L. intracellularis in said biological sample, and wherein the absence of said polypeptide-antibody complex indicates the absence of L. intracellularis in said biological sample.

25. The method of claim 24, wherein said polypeptide is attached to a solid support.

26. The method of claim 24, wherein said biological sample is selected from the group consisting of a fecal sample and a blood sample.

27. A method for detecting the presence or absence of L. intracellularis in a biological sample, comprising the steps of:
contacting said biological sample with the antibody of claim 14, wherein an antibody-polypeptide complex is produced if a polypeptide is present in said biological sample for which said antibody has specific binding affinity, and
detecting the presence or absence of said antibody-polypeptide complex,
wherein the presence of said antibody-polypeptide complex indicates the presence of L. intracellularis in said biological sample, and wherein the absence of said antibody-polypeptide complex indicates the absence of L. intracellularis in said biological sample.

28. The method of claim 27, wherein said antibody is bound to a solid support.

29. The method of claim 27, wherein said biological sample is selected from the group consisting of a blood sample or a milk sample.

30. A method of preventing infection by L. intracellularis in an animal, comprising the steps of:
administering a compound to said animal, wherein said compound comprises the polypeptide of claim 11,
wherein said compound immunizes said animal against L. intracellularis.

31. A method of preventing infection by L. intracellularis in an animal, comprising the steps of:
administering a compound to said animal, wherein said compound comprises a nucleic acid, wherein said nucleic acid comprises a nucleic acid molecule of at least 10 nucleotides in length, said molecule having at least 75% sequence identity to SEQ ID NO:8741,
wherein said compound immunizes said animal against L. intracellularis.

32. A composition comprising a first oligonucleotide primer and a second oligonucleotide primer, wherein said first oligonucleotide primer and said second oligonucleotide primer are each 10 to 50 nucleotides in length, and wherein said first and second oligonucleotide primers, in the presence of L. intracellularis nucleic acid, generate an amplification product under standard amplification conditions, but do not generate an amplification product in the presence of nucleic acid from an organism other than L. intracellularis.

33. An isolated nucleic acid, wherein said nucleic acid comprises a nucleic acid molecule greater than 10 nucleotides in length, said molecule having at least 75% sequence identity to SEQ ID NO:8741 or to the complement of SEQ ID NO:8741, wherein said molecule hybridizes under stringent conditions with L. intracellularis nucleic acid but does not hybridize with nucleic acid from an organism other than L. intracellularis under the same hybridization conditions.

34. An article of manufacture, wherein said article of manufacture comprises the composition of claim 32.

35. An article of manufacture, wherein said article of manufacture comprises the isolated nucleic acid of claim 1.
